(19) United States
(12) Patent Application Publication
(10) Pub. No.: US 2016/0370352 A1
(43) Pub. Date: Dec. 22, 2016

(54) ASSAYS AND MONITORING PARADIGMS FOR STEM CELL CULTURE

(71) Applicant: MERCK PATENT GMBH, Darmstadt (DE)

(72) Inventors: Julie Murrell, Westford, MA (US); Martha Rook, Harvard, MA (US); Ellen Binder, Darmstadt (DE); Nikolai Stankiewicz, Darmstadt (DE)

(21) Appl. No.: 14/898,809

(22) PCT Filed: Dec. 4, 2014
(86) PCT No.: PCT/EP2014/003234

§ 371 Date: Dec. 16, 2015

Related U.S. Application Data
(60) Provisional application No. 61/915,880, filed on Dec. 13, 2013.

(51) Int. Cl. G01N 33/50; A61K 35/32; A61K 35/35; C12Q 1/68; A61K 35/28

(52) U.S. Cl. CPC G01N 33/5073; C12Q 1/6881; A61K 35/28; A61K 35/35; A61K 35/32; G01N 2333/47

(57) ABSTRACT

The invention provides, inter alia, methods for monitoring the differentiation state of cultured cells, such as cultured MSCs, as well as related kits and systems for performing the methods. In certain embodiments, the methods entail measuring the level of one or more of KLF5, FABP4, Adiponectin/ADIPOQ, Chemerin/RARRES2, KLF4, PAI1, Runx2, ALPL, Osteocalcin/BGLAP, CTNNB1, Osteonectin/SPARC, Sox9, COL2A1, MIA, and COMP.

This application claims the benefit of priority of U.S. provisional patent application No. 61/915,880, filing date Dec. 13, 2014, the entire content of which is incorporated herein by reference.

### BACKGROUND OF THE INVENTION

Mesenchymal stem cells (MSCs) are of immense interest for, inter alia, regenerative medicine. Current methods to characterize MSC differentiation by immunohistochemical means are elaborate (multi-step), time-consuming (>3 weeks), and affected by subjective evaluation (operator dependent readout on a microscope). There is a need for reproducible, scalable cell processing capabilities and robust, validated monitoring and characterization assays. Current differentiation assays are so time intensive that they cannot be considered for a monitoring and characterization application.

### SUMMARY OF THE INVENTION

The invention provides, inter alia, methods for monitoring the differentiation state of cultured cells, such as cultured MSCs, as well as related kits and systems for performing the methods.

In a first aspect, the invention provides methods of monitoring the differentiation state of a mesenchymal stem cell (MSC) culture. These methods entail measuring the expression level of one or more markers of adipogenesis, one or more markers of osteogenesis, or one or more markers of chondrogenesis in an isolated sample from the culture, where a change in the expression level of one or more of the markers, relative to a suitable control, indicates a change in the differentiation state of the MSC culture.

In particular embodiments the one or more markers of adipogenesis is selected from KLF5, FABP4, Adiponectin/ADIPOQ, Chemerin/RARRES2, KLF4, and PAI1; the one or more markers of osteogenesis is selected from Runx2, ALPL, Osteocalcin/BGLAP, CTNNB1, and Osteonectin/SPARC; or c) the one or more markers of chondrogenesis is selected from Sox9, COL2A1, MIA, and COMP. In more particular embodiments, the expression level of one or more markers of adipogenesis, one or more markers of osteogenesis, and one or more markers of chondrogenesis are measured.

In certain embodiments, the one or more markers of adipogenesis are selected from KLF5, FABP4, and PAI1; more particularly, the expression levels of two or all three of KLF5, FABP4, and PAI1 are measured; still more particularly, the expression levels of KLF5, FABP4, and PAI1 are measured; yet more particularly, the expression level of one or more of Adiponectin/ADIPOQ, Chemerin/RARRES2, and KLF4 (e.g., two, three, or all four additional expression levels) are measured in addition to KLF5, FABP4, or PAI1; and, still more particularly, the expression levels of KLF5, FABP4, Adiponectin/ADIPOQ, Chemerin/RARRES2, KLF4, and PAI1 are measured.

In some embodiments, the one or more markers of osteogenesis are selected from Runx2, ALPL, and CTNNB1; more particularly, the expression levels of two or all three of Runx2, ALPL, and CTNNB1 are measured; still more particularly, the expression levels of Runx2, ALPL, and CTNNB1 are measured; more particularly, the expression level of one or more of Osteocalcin/BGLAP and Osteonectin/SPARC is measured in addition to Runx2, ALPL, and CTNNB1; yet more particularly, the expression levels of Runx2, ALPL, Osteocalcin/BGLAP, CTNNB1, and Osteonectin/SPARC are measured.

In certain embodiments, the expression levels of two, three, or all four of Sox9, COL2A1, MIA, and COMP are measured; more particularly, the expression levels of Sox9, COL2A1, MIA, and COMP are measured. In some embodiments, the one or more markers of chondrogenesis are measured, which are selected from Sox9, MIA, COMP and COLA1.

In particular embodiments, the expression levels of KLF5, FABP4, PAI1 Runx2, ALPL, and CTNNB1 are measured; more particularly, the expression levels of KLF5, FABP4, PAI1 Runx2, ALPL, CTNNB1, Sox9, COL2A1, MIA, and COMP are measured; still more particularly, the expression levels of KLF5, FABP4, Adiponectin/ADIPOQ, Chemerin/RARRES2, KLF4, PAI1 Runx2, ALPL, Osteocalcin/BGLAP, CTNNB1, Osteonectin/SPARC, Sox9, COL2A1, MIA, and COMP are measured.

In some embodiments of any of preceding aspects and embodiments, the monitoring of the differentiation state of the MSC culture provides a quality control assay. In more particular embodiments, the method optionally further comprises monitoring (by the methods provided by the invention) or performing the step of harvesting MSCs from the culture; or may further comprise monitoring or performing the step of making a cell bank from the MSC culture; or monitoring or making the final cell product.

In any of the preceding aspects and embodiments, the MSC culture is induced to differentiate along a lineage selected from adipocyte, chondroblast, or osteoblast.

For some embodiments of any of the preceding aspects and embodiments, the expression levels of the one or more markers are measured simultaneously. In other embodiments, the expression levels of the one or more markers are measured sequentially.

In some embodiments of any of the preceding aspects and embodiments, the expression levels are measured at the protein level. In particular embodiments, the protein expression levels are measured optical, mechanical, acoustic, thermal or physical methods; an immunoassay, Western blotting, ELISA (enzyme-linked immunosorbent assay), MSIA (mass spectrometric immunoassay), MS/MS (tandem mass spectrometry), RIA (radioimmunoassay), peptide sequencing, flow cytometry, surface plasmon resonance, aptamer-based assay, multiplexing (e.g., LUMINEX® XMAP®), bead based detection systems, spectroscopic methods, interferometry, chromatographic methods, fluorescent methods, colorimetric methods, luminescent methods, magnetic methods, electrical methods, piezoelectrical methods, electrochemical read-out systems, HPLC, or NMR-based technologies In more particular embodiments, the expression levels are measured using an immunoassay. In still more particular embodiments, the immunoassay is a sandwich immunoassay using, for each marker, a pair of detectably labeled binders that specifically bind the marker. In yet more particular embodiments, the binders are fluorescently labeled and the expression levels are measured by flow cytometry. In certain embodiments where levels of markers are measured at the protein level, the protein expression levels are measured from the culture supernatant.

In other embodiments of any of the preceding aspects and embodiments, the expression levels are measured at the nucleic acid level. In particular embodiments, the nucleic acid expression levels are measured by quantitative polymerase chain reaction (qPCR), quantitative real-time polymerase chain reaction (qRTPCR), digital droplet PCR, (ddPCR), SAGE (serial analysis of gene expression), sequencing, northern blotting, microarrays, transcription mediated amplification, isothermal amplification or Southern blotting.

In particular embodiments of any one of the preceding aspects and embodiments, the expression levels are measured within, relative to obtaining the sample, about: 0 hours, 0.5 hours, 1 hour, 2 hours, 3 hours, 4 hours, 5 hours, 6 hours, 12 hours, 18 hours, or 24 hours; or about: 1 day, 2 days, 3 days, 4 days, or 5 days.

In certain embodiments of any one of the preceding aspects and embodiments, the sample is obtained, for measuring the expression levels, at about: 0, 5, 10, 15, 30, 45, 60, 75, or 90 minutes; or 2, 3, 4, 5, 6, 12, 18, or 24 hours; or 1, 2, 3, 4, 5, 6, 7, 8, 9, 10, 12, 14, 21, or 28 days, of starting the culture.

In particular embodiments of any one of the preceding aspects and embodiments, the expression levels are measured multiple times in a time series, e.g., at least: 2 times, 3 times, 4 times, 5 times, 6 times, 7 times, 8 times, 9 times, or 10 times. In some embodiments, the multiple measurements are made over the span of the culture (from inoculation, through harvesting and further processing), e.g., over a period of about: 1 day, 2 days, 3 days, 4 days, 5 days, 6 days, or 7 days, or about: 1 week, 2 weeks, 3, or 4 weeks, or more.

In some embodiments, the sample from the culture comprises both cells and culture medium. In particular embodiments, the cells in the sample are lysed before measuring the expression levels. In more particular embodiments, the cells in the sample are lysed by mechanical/physical methods, enzymatic methods, or chemical methods.

In certain embodiments of any of the preceding aspects and embodiments, MSC culture is a monolayer on a planar solid surface. In other embodiments of any of the preceding aspects and embodiments, the MSC culture is a liquid suspension culture. In particular embodiments, the liquid suspension culture comprises growing the cells on a microcarrier. In more particular embodiments, the microcarrier comprises dextran, collagen, polystyrene, glass, polymers, agarose or a combination thereof. In more particular embodiments, the microcarrier is a bead formed of porous glass.

In certain embodiments of any of the preceding aspects and embodiments, the liquid suspension culture is in a bioreactor. In particular embodiments, the bioreactor has a capacity of about: 1, 10, 50, 100, 250, 500, 750 mL or more, e.g., 1 L, 3 L, 5 L, 15 L, 50 L, 100 L, 250 L, 500 L, 1000 L, 5000 L, 10000 L, 20000 L, 30000 L, 40000 L, or 50000 L, or more. In more particular embodiments, the MSCs are cultured by batch, batch refeed, fed batch, partial medium exchange or perfusion.

In certain embodiments of any of the preceding aspects and embodiments, the MSCs are mammalian MSCs. In particular embodiments, the mammalian MSCs are human MSCs.

In some embodiments of any of the preceding aspects and embodiments, the culture is a working cell bank or a master cell bank, e.g., derived from a donor.

In certain embodiments of any of the preceding aspects and embodiments, the methods further include the step of measuring the expression level of one or more markers of myogenesis, including cardiogenesis, or one or more markers of neurogenesis, one or more markers of kidney differentiation, or any tissue or organ of the mesodermal, endodermal, or ectodermal lineage. In more particular embodiments, the one or more additional markers can be for any tissue or organ of the mesodermal or ectodermal lineage.

In some embodiments of any of the preceding aspects and embodiments, the methods entail determining if the cells in a sample of the MSC culture: i) adhere to plastic in standard culture conditions; ii) meet the condition that more than about: 80%, 85%, 90%, 95%, or more, of the cells express one or more of CD105, CD73, or CD90, more preferably, wherein 95% or more of the cells express one or more of CD105, CD73, or CD90, still more preferably, wherein 95% or more of the cells express one or more of CD105, CD73, and CD90; iii) meet the condition that less than about: 10%, 9%, 8%, 7%, 6%, 5%, 4%, 3%, 2%, 1%, or fewer, of the cells express one or more of CD45; CD34; CD14 or CD11 b; CD79a or CD19; or HLA-DR, more preferably, wherein 2% or fewer of the cells express one or more of CD45; CD34; CD14 or CD11 b; CD79a or CD19; or HLA-DR, still more preferably, wherein 2% or fewer of the cells express CD45; CD34; CD14 or CD11b; CD79α or CD19; and HLA-DR; iv) are capable of differentiating along adipocyte, chondroblast, or osteoblast lineages under standard in vitro differentiating conditions; or v) 1, 2, 3, or all 4 of i), ii), iii), and iv).

In some embodiments, the methods provided by the invention further entail the step of visualizing the morphology of cells in a sample from the culture. In particular embodiments, the visualization is to classify the differentiation state of the cells by their morphology.

In certain embodiments, the methods provided by the invention further include the step of providing a user-readable display of the measured expression levels. In particular embodiments, the display is in the form of one or more graphs of the expression levels. In more particular embodiments, the one or more graphs of the expression levels are of a time series of expression levels. In yet more particular embodiments, the display provides a summary of the differentiation state of the culture.

In another aspect, the invention provides methods of treating a subject in need of cells selected from MSCs, adipocytes, chondroblasts, or osteoblasts. These methods entail providing the subject a therapeutically effective amount of the cells from a culture tested by the method of any of the aspects and embodiments described in the application and determined to contain cells in the necessary differentiation state.

In yet another aspect, the invention provides kits suitable for performing the methods of any one of the methods provided by the invention. In particular embodiments, the kits contain reagents for detecting the expression level of: i) one or more markers of adipogenesis selected from KLF5, FABP4, Adiponectin/ADIPOQ, Chemerin/RARRES2, KLF4, and PAI1; ii) one or more markers of osteogenesis selected from Runx2, ALPL, Osteocalcin/BGLAP, CTNNB1, and Osteonectin/SPARC; iii) one or more markers of chondrogenesis selected from Sox9, COL2A1, MIA, and COMP; or iv) or any combination of the 15 markers. In more particular embodiments, the reagents are detectably labeled and suitable for the simultaneous singleplex or multiplex detection of the one or more markers. In still more particular embodiments, the reagents are, for each marker, a pair of detectably labeled antibodies that specifically bind the marker. In yet more particular embodiments, the antibodies are fluorescently labeled and suitable for simultaneous multiplex detection.

In a further aspect, the invention provides non-transient computer-readable media with instructions that, if executed by a processor, would cause the processor to perform steps comprising: accepting data representing the expression levels of: i) one or more markers of adipogenesis selected from KLF5, FABP4, Adiponectin/ADIPOQ, Chemerin/RARRES2, KLF4, and PAI1; and/or ii) one or more markers of osteogenesis selected from Runx2, ALPL, Osteocalcin/BGLAP, CTNNB1, and Osteonectin/SPARC; and/or iii) one or more markers of chondrogenesis selected from Sox9, COL2A1, MIA, and COMP; or any combination comprising any of the 15 markers; and evaluating the expression level of one or more of the markers, where a change in the expression levels, relative to a suitable control, indicates a change in the differentiation state of the MSC culture. In some embodiments, the computer-readable medium is suitable for performing any one of the methods provided by the invention.

In a related aspect, the invention provides a system comprising a computer-readable media provided by the invention and a processor for executing the instructions. In particular embodiments, the system includes a user-readable display for displaying the measured gene expression levels.

### BRIEF DESCRIPTION OF THE DRAWINGS

The foregoing will be apparent from the following more particular description of example embodiments of the invention, as illustrated in the accompanying drawings.

FIGS. 1A-1B provide bar graphs illustrating the expression level of adipocyte markers provided by the invention. In FIG. 1A, MFI signals for the different adipocyte lineage markers are shown for different time-points of the differentiation. Cell samples were taken on the respective days of the differentiation procedure and processed following the MILLIPLEX® MAP Assay Protocol. FIG. 1B is a fingerprint of MFI data showing the sequential expression of the adipocyte differentiation markers, where A is KLF4, B is KLF5, C is PAI1 D is FABP4, E is Chemerin, and F is ADIPOQ.

FIGS. 2A-2B provide bar graphs illustrating the expression level of osteoblast markers provided by the invention. In FIG. 2A, MFI signals for the different osteoblast lineage markers are shown for different time-points of the differentiation. Cell samples were taken on the respective days of the differentiation procedure and processed following the MILLIPLEX® MAP Assay Protocol. *Only for the Osteonectin cell culture supernatants were used. FIG. 2B is a fingerprint of MFI data showing the sequential expression of the osteoblast differentiation markers, where M is Runx2, N is ALPL, 0 is Osteonectin*, P is Osteocalcin and Q is Beta-Catenin.

FIGS. 3A-3B provide bar graphs illustrating the expression level of chondrocyte markers provided by the invention. In FIG. 3A, MFI signals for the different chondrocyte lineage markers are shown for different time-points of the differentiation. Cell samples were taken on the respective days of the differentiation procedure and processed following the MILLIPLEX® MAP Assay Protocol. FIG. 3B is a fingerprint of MFI data showing the sequential expression of the osteoblast differentiation markers, where R is SOC9, S is MIA, T is COMP, and U is COL2A1.

FIG. 4 is a graph of PAD levels over time. PAI1 is an early marker of adipogenesis and is expressed in undifferentiated hMSCs. PAI1 was expressed over the course of the bioreactor campaign, with an MFI of 2000, maximally. The pattern of expression increased over the 2 week run. Undifferentiated hMSCs expressed PAI1 at a level 9000 MFI.

FIG. 5 is a graph of Klf5 levels over time. Klf5 is an early marker of adipogenesis and is expressed in undifferentiated hMSCs. Klf5 was expressed over the course of the bioreactor campaign at an extremely low level (50 MFI), similar to what was seen in undifferentiated hMSCs (75 MFI).

FIG. 6 is a graph of FABP4 levels over time. FABP4 is an intermediate to late marker of adipogenesis and is not expressed in undifferentiated hMSCs. FABP4 was expressed over the course of the bioreactor campaign, with an MFI of 500. Undifferentiated hMSCs expressed PAI1 at a level 350 MFI. Differentiated hMSCs have a high level of FABP4 (3000 MFI).

FIG. 7 is a graph of Runx2 levels over time. Runx2 is an early marker of osteogenesis and is expressed in undifferentiated hMSCs. Runx2 was expressed over the course of the bioreactor campaign, with an MFI of 1500, maximally. The pattern of expression was relatively constant over the 2 week run. Undifferentiated hMSCs expressed Runx2 at a level 4000 MFI.

FIG. 8 is a graph of Beta-catenin levels over time. Beta-catenin is an early marker of osteogenesis and is expressed in undifferentiated hMSCs. Beta-catenin was expressed at a constant level over the course of the bioreactor campaign (2500 MFI), similar to what was seen in undifferentiated hMSCs (1500 MFI).

FIG. 9 is a graph of ALPL levels over time. ALPL is an intermediate to late marker of osteogenesis and is not expressed in undifferentiated hMSCs. ALPL was not expressed at any point during the bioreactor campaign. Differentiated hMSCs have a high level of ALPL (6000 MFI).

FIG. 10 is a graph of Sox 9 levels over time. Sox9, an early marker of chondrogenesis is expressed in cells grown in bioreactors and undifferentiated cells at the same levels, indicating that the bioreactor does not cause the progression through chondrocyte differentiation pathway.

### DETAILED DESCRIPTION OF THE INVENTION

A description of example embodiments of the invention follows.

### Monitoring Differentiation State of MSCs

The invention provides methods of monitoring the differentiation state of a mesenchymal stem cell (MSC) culture comprising measuring the expression level of one or more markers of adipogenesis, and/or one or more markers of osteogenesis, and/or one or more markers of chondrogenesis.

“Differentiation state” is the phenotype of an MSC that illustrates its ability to differentiate along different lineages, including, but not limited to, adipocyte, chondroblast, and osteoblast lineages. A “change in the differentiation state” of an MSC can be detected as a change in the expression level of one or more markers provided by the invention and can include, for example, increased or decreased expression level of an early marker of differentiation or increased expression of an intermediate or late marker of at least one of adipogenesis, chondrogenesis, or osteogenesis, as illustrated by the accompanying working examples. Accordingly, in some embodiments, a change in differentiation state can be detected by a qualitative presence or absence of one or more markers. A change in the differentiation state can be measured at a single time point (e.g., expression of an intermediate or late marker at a single time point could indicate differentiation of the MSC culture, as could, e.g., the loss of an early marker) or over time by measuring one or more expression levels at multiple time points and observing changes in the expression level of one or more markers provided by the invention.

“Mesenchymal stem cell,” “MSC,” and the like refer to multipotent stromal stem cells, also known as mesenchymal stromal cells, multipotent stromal cells, multipotent stem cells, and mesenchymal stromal/stem cells. Exemplary criteria for identifying MSCs are described in, for example, Dominici, et al., Cytotherapy 8(4): 315-317 (2006), which is incorporated by reference in its entirety. In some embodiments, MSCs are characterized by: i) the ability to adhere to plastic in standard culture conditions; ii) more than about: 80%, 85%, 90%, 95%, or more, of the cells expressing one or more of CD105, CD73, or CD90 (e.g., 95% or more of the cells express all three markers); iii) less than about: 10%, 9%, 8%, 7%, 6%, 5%, 4%, 3%, 2%, 1%, or fewer, of the cells expressing one or more markers, such as CD45; CD34; CD14 or CD11b; CD79a OR CD19; or HLA-DR (e.g., 2% or fewer of the cells express any one of CD45; CD34; CD14 or CD11b; CD79α OR CD19; or HLA-DR; more preferably, 2% or fewer of the cells express CD45; CD34; CD14 or CD11 b; CD79a or CD19; or HLA-DR); iv) the capability to differentiate along adipocyte, chondroblast, and osteoblast lineages under standard in vitro differentiation conditions (as described in, for example, the Exemplification below), or a combination of the foregoing. In more particular embodiments, MSCs adhere to plastic in standard culture conditions, display the appropriate signature of markers described above (both positive and negative staining, at the indicated levels, such as at least 95% for the indicated positive markers and less than 2% positive for the negative markers), and are capable of differentiating along adipocyte, chondroblast, and osteoblast lineages under standard in vitro differentiation conditions. Exemplary differentiation conditions are described in the Exemplification section, below.

MSCs can be from any source, including adult, youth, neonatal, or fetal sources, and can be from either humans or non-human animals. Any suitable tissue can be a source of MSCs, provided that vasculature exists in the sample. Exemplary tissue sources of MSCs include, for example, bone marrow, adipose, placenta, or umbilical cord. Any species with MSCs can be a source of the MSCs for use consonant with the invention, and, in particular embodiments, the MSCs are mammalian MSCs, and, in more particular embodiments, the MSCs are human MSCs. The cultured cells can be cells for a master cell bank or working cell bank (i.e., to be expanded and then preserved), or cells from a master cell bank or a working cell bank.

A “master cell bank” serves the long term storage and preservation of the respective cells (e.g., a source of cells, e.g., from a patient) and is usually stored at −70° C. or lower and preferably in liquid nitrogen/at liquid nitrogen temperatures. To make a master cell bank, a culture of cells is distributed into containers in a single operation, processed together in such a manner as to ensure uniformity, and stored in such a manner as to ensure stability.

A “working cell bank” serves the short term storage and preservation of the respective cells and is usually stored at −70° C. or lower and preferably in liquid nitrogen/at liquid nitrogen temperatures. A working cell bank is a culture of cells derived from the master cell bank and intended for use in the preparation of production cell cultures. A culture of the cells is distributed into containers in a single operation, processed together in such a manner as to ensure uniformity and stored in such a manner as to ensure stability.

“Culture” (as in an MSC culture), “culturing,” and the like refer to the in vitro growth, expansion, or maintenance of cells and include, in some embodiments, growth in controlled media and other environmental conditions, such as temperature, pH, osmolarity, et cetera. “Culture” can include both bench scale growth in, e.g., Petri dishes or T-flasks, as well as bioreactors (including stirred tank and suspension bioreactors) and liquid suspension growth on either laboratory or large production scales—with or without microcarriers. An “MSC culture” is a population of MSCs grown in culture. A “sample,” e.g., of a culture, is a portion of the culture that is representative of the whole. Cultures that can be analyzed by the methods provided by the invention can, in their cellular component, comprise, consist essentially of, or consist of MSC, e.g., in an undifferentiated state, or comprise, consist essentially of, or consist of, in their cellular component, cells in varying degrees of differentiation along any lineage (e.g. adipogenesis, osteogenesis, or chondrogenesis).

In particular embodiments, the MSC culture is a monolayer on a planar solid surface. The planar surface can be porous or substantially non-porous. In other embodiments, the MSC culture is a liquid suspension culture—with or without a microcarrier. Cells can be single-cell suspensions, aggregates, or a combination thereof. In more particular embodiments, the MSC culture is a liquid suspension culture (e.g., in a stirred or otherwise mixed tank), optionally comprising growing the cells on a microcarrier. The microcarrier comprises any material that can act as a substrate for cell growth or maintenance, including, but not limited to, dextran, collagen, polystyrene, glass, polymers, agarose, or a combination thereof. Microcarriers for use in the invention can be any suitable form including regular or irregular in form, beads, oblong-shaped (e.g., winged fiber cut very short), extruded materials, or 3-D scaffolds. Microcarriers can be porous or substantially non-porous.

In more particular embodiments, the liquid suspension culture is in a bioreactor. The bioreactor can be any capacity, such as about: 1, 10, 50, 100, 250, 500, 750 mL or more, e.g., 1 L, 3 L, 5 L, 15 L, 50 L, 100 L, 250 L, 500 L, 1000 L, 5000 L, 10000 L, 20000 L, 30000 L, 40000 L, or 50000 L, or more. The bioreactor can culture by any suitable method, such as batch, batch refeed, fed batch, partial medium exchange, or perfusion.

### Markers Provided by the Invention

“Marker(s),” such as “marker(s) of adipogenesis,” “marker(s) of osteogenesis,” “marker(s) of chondrogenesis,” and the like, are gene expression products that are informative of the differentiation state of a cell, more particularly, an MSC; e.g., they are a molecular phenotypes that are illustrative of the MSC's differentiation state. “Markers provided by the invention” include the “marker(s) of adipogenesis” (e.g., CEBP, CMKLR1, PPARG2, CEBPA, MED1, KLF5, FABP4, Adiponectin/ADIPOQ, Chemerin/RARRES2, KLF4, and PAI1; more particularly, KLF5, FABP4, Adiponectin/ADIPOQ, Chemerin/RARRES2, KLF4, and PAI1; still more particularly, KLF5, FABP4, and PAI1), “marker(s) of osteogenesis” (e.g., MSX2, Osterix/SP7, COL1A1, Osteopontin/SPP1, Runx2, ALPL, Osteocalcin/BGLAP, CTNNB1, and Osteonectin/SPARC; more particularly, Runx2, ALPL, Osteocalcin/BGLAP, CTNNB1, and Osteonectin/SPARC; still more particularly, Runx2, ALPL, and CTNNB1), and “marker(s) of chondrogenesis” (e.g., Sox6, Sox5, MATN1, COL9A1, ACAN, Sox9, COL2A1, MIA, and COMP; more particularly, Sox9, COL2A1, MIA, and COMP) described in this application.

Various combinations of the markers provided by the invention can be used together consonant with the invention. For example, where levels of one or more markers of adipogenesis are measured, levels of 1, 2, 3, 4, 5, 6, 7, 8, 9, 10, or all 11 of CEBP, CMKLR1, PPARG2, CEBPA, MED1, KLF5, FABP4, Adiponectin/ADIPOQ, Chemerin/RARRES2, KLF4, and PAI1 are measured. In more particular embodiments, levels of 1, 2, 3, 4, 5, or all 6 of KLF5, FABP4, Adiponectin/ADIPOQ, Chemerin/RARRES2, KLF4, and PAI1 are measured. In still more particular embodiments, levels of 1, 2, or all 3 of KLF5, FABP4, and PAI1 are measured. Where levels of one or more markers of osteogenesis are measured, levels of 1, 2, 3, 4, 5, 6, 7, 8, or all 9 of MSX2, Osterix/SP7, COL1A1, Osteopontin/SPP1, Runx2, ALPL, Osteocalcin/BGLAP, CTNNB1, and Osteonectin/SPARC are measured. In more particular embodiments, levels of 1, 2, 3, 4, or all 5 of Runx2, ALPL, Osteocalcin/BGLAP, CTNNB1, and Osteonectin/SPARC are measured. In still more particular embodiments, levels of 1, 2, or all 3 of Runx2, ALPL, and CTNNB1 are measured. Similarly, where levels of one or more markers of chondrogenesis are measured, levels of 1, 2, 3, 4, 5, 6, 7, 8, or all 9 of Sox6, Sox5, MATN1, COL9A1, ACAN, Sox9, COL2A1, MIA, and COMP are measured. In more particular embodiments, levels of 1, 2, 3, or all 4 of Sox9, COL2A1, MIA, and COMP are measured.

Various combinations of markers of adipogenesis (e.g., from 1 to 11, 1 to 6, or 1 to 3, as described above), markers of osteogenesis (e.g., from 1 to 9, 1 to 5, or 1 to 3, as described above), and markers of chondrogenesis (e.g., from 1 to 9 or 1 to 4, as described above) can be measured in the methods provided by the invention. For example, levels of any set of markers of adipogenesis can be measured with levels of any set of markers of osteogenesis or levels of any set of markers of chondrogenesis. Also, levels of any set of markers of osteogenesis can be measured with levels of any set of markers of chondrogenesis. Naturally, levels of any set of markers of adipogenesis can be measured with levels of any set of markers of osteogenesis and levels of any set of markers of chondrogenesis (e.g., any combination of 3 to 29 markers, provided there is at least one marker for each of adipogenesis, osteogenesis, and chondrogenesis).

Table A, below, provides NCBI human genelDs and RefSeq mRNA and protein sequences for the markers provided by the invention. Where multiple isoforms of the RefSeqs are available, isoform 1 is presented as an example. These identifiers may be used to retrieve, inter alia, publicly-available annotated mRNA or protein sequences from sources such as the NCBI website, which may be found at the following uniform resource locator (URL): //www.ncbi.nlm.nih.gov. The information associated with these identifiers, including reference sequences and their associated annotations, are all incorporated by reference. Additional useful tools for converting IDs or obtaining additional information on a gene are known in the art and include, for example, DAVID, Clone/GeneID converter and SNAD. See Huang et al., Nature Protoc. 4(1):44-57 (2009), Huang et al., Nucleic Acids Res. 37(1):1-13 (2009), Alibes et al., BMC Bioinformatics 8:9 (2007), Sidorov et al., BMC Bioinformatics 10:251 (2009).

TABLE A

| Marker | Gene | RefSeq |
|---|---|---|
| Adipogenesis | | |
| KLF5 | 688 | NM_001730.4 → NP_001721.2 |
| FABP4 | 2167 | NM_001442.2→ NP_001433.1 |
| ADIPOQ | 9370 | NM_001177800.1→ NP_001171271.1 |
| RARRES2 | 5919 | NM_002889.3 → NP_002880.1 |
| KLF4 | 9314 | NM_004235.4 → NP_004226.3 |
| PAI1/ | 5054 | NM_000602.4 → NP_000593.1 |
| SERPINE1 | | |
| CEBP | 64506 | NM_030594.3 → NP_085097.3 |
| CMKLR1 | 1240 | NM_001142343.1 → NP_001135815.1 |
| PPARG2 | 5468 | NM_138712.3 → NP_619726.2 |
| CEBPA | 1050 | NM_004364.4 → NP_004355.2 |
| MED1 | 5469 | NM_004774.3 → NP_004765.2 |
| Osteogenesis | | |
| Runx2 | 860 | NM_001024630.3 → NP_001019801.3 |
| ALPL | 249 | NM_000478.4 → NP_000469.3 |
| BGLAP | 632 | NM_199173.4 → NP_954642.1 |
| CTNNB1 | 1499 | NM_001904.3 → NP_001895.1 |
| SPARC | 6678 | NM_003118.3 → NP_003109.1 |
| MSX2 | 4488 | NM_002449.4 → NP_002440.2 |
| SP7 | 121340 | NM_001173467.1 → NP_001166938.1 |
| COL1A1 | 1277 | NM_000088.3 → NP_000079.2 |
| SPP1 | 6696 | NM_001040058.1 → NP_001035147.1 |
| Chondrogenesis | | |
| Sox9 | 6662 | NM_000346.3 → NP_000337.1 |
| COL2A1 | 1280 | NM_001844.4 → NP_001835.3 |
| MIA | 8190 | NM_006533.3 → NP_006524.1 |
| COMP | 1311 | NM_000095.2 → NP_000086.2 |
| Sox6 | 55553 | NM_017508.2 → NP_059978.1 |
| Sox5 | 6660 | NM_006940.4 → NP_008871.3 |
| MATN1 | 4146 | NM_002379.3 → NP_002370.1 |
| COL9A1 | 1297 | NM_001851.4 → NP_001842.3 |
| ACAN | 176 | NM_001135.3 → NP_001126.3 |

“Expression level” is the amount of a gene expression product (e.g., a marker provided by the invention) and can encompass both nucleic acid (e.g., mRNA, miRNA) and protein gene expression products. Expression levels can be absolute (or relative) measures and may be optionally normalized by any means (e.g., as percentage of maximal values, mean/variance normalized, relative expression to a reference gene expression product or relative to a reference time), or transformed by any means (e.g., log transformed, using any suitable base, e.g., base 2, base 10, base e).

“Measuring” an expression level, such as a marker provided by the invention, requires contacting a sample with isolated analytic tools that are a product of man, such as laboratory equipment for measuring the level, and, in certain embodiments, additional isolated reagents, such as isolated oligonucleotides, microarrays, sequencing reagents (such as cloned enzymes, detectably labeled dNTPs, et cetera), or binders (as described below, which may be recombinantly produced and/or detectably labeled) to measure the level of a gene expression product by an analytical laboratory method. In particular embodiments, the detectably labeled reagents (such as antibodies or nucleic acids, such as synthetic oligonucleotides) are artificially and/or detectably labeled—i.e., the reagents are products of man that do not exist in nature. Measuring the level of a gene expression product can be done directly in the course of the analytical laboratory methods or, in some embodiments, by evaluating the quantitative output of the analytical laboratory methods.

A “suitable control” includes, for example, an earlier or later time point in a time series (e.g., when the differentiation state of the cells in the culture was known), and a culture grown in parallel or in series (either before or after) to the culture being analyzed, as well as reference values previously compiled from samples determined—by any means—to be in a particular differentiation state. For example, reference values for one or more markers may be compiled and used to develop a binary or probabilistic classification algorithm that is then used to classify the differentiation state of a sample, and the use of such classification algorithms therefore entails comparison to suitable controls. Expression levels (e.g., for one or more markers) can be evaluated and classified by a variety of means such as general linear model (GLM), ANOVA, regression (including logistic regression), support vector machines (SVM), linear discriminant analysis (LDA), principal component analysis (PCA), k-nearest neighbor (INN), neural network (NN), nearest mean/centroid (NM), and Bayesian covariate predictor (BCP). A classification model can be developed using any of the subsets and combinations of markers described herein based on the teachings of the invention. Suitable cutoffs for evaluating an expression level, such as a panel, (e.g., for classification in a particular differentiation state) can be determined using routine methods, such as ROC (receiver operating characteristic) analysis, and may be adjusted to achieve the desired sensitivity (e.g., at least about 50, 52, 55, 57, 60, 62, 65, 67, 70, 72, 75, 77, 80, 82, 85, 87, 90, 92, 95, 97, or 99% sensitivity) and specificity (e.g., at least about 50, 52, 55, 57, 60, 62, 65, 67, 70, 72, 75, 77, 80, 82, 85, 87, 90, 92, 95, 97, or 99% specificity).

Expression levels of markers provided by the invention can be measured at the nucleic acid level, the protein level, or both the nucleic acid and protein level. Expression levels can be measured from any suitable sample, such as a sample comprising cells and/or supernatant. When the sample comprises cells, in some embodiments, the cells are lysed before measuring the expression levels. In particular embodiments, the cells are lysed by mechanical/physical methods (including, but not limited to, sonication, boiling, homogenization, freezing, and bead mill), enzymatic methods, chemical methods, or a combination thereof.

Nucleic acid gene expression products can be measured by any suitable means. In particular embodiments, nucleic acid expression levels are measured by quantitative polymerase chain reaction (qPCR), quantitative real-time polymerase chain reaction (qRTPCR), digital droplet PCR, (ddPCR), SAGE (serial analysis of gene expression), sequencing (including next-generation sequencing, such as sequencing by synthesis, pyrosequencing, dideoxy sequencing, and sequencing by ligation, or any other methods known in the art, such as discussed in Shendure et al., Nat. Rev. Genet. 5:335-44 (2004) or Nowrousian, Euk. Cell 9(9): 1300-1310 (2010), including such specific platforms as HELICOS®, ROCHE® 454, ILLUMINA®/SOLEXA®, ABI SOLiD®, and POLONATOR® sequencing), northern blotting, microarrays, transcription mediated amplification, isothermal amplification, or Southern blotting.

Expression levels can be determined by measuring and/or testing the reference nucleic acid sequences listed in Table A—as well as complements, fragments, and similar nucleic acid sequences of the reference nucleic acid sequences listed in Table A-including any combination described in the application. “Similar nucleic acid sequences” can be naturally occurring (e.g., allelic variants or homologous sequences from other species) or engineered variants relative to the reference nucleic acid sequences in Table A and, in some embodiments, will be at least about 60, 65, 70, 75, 80, 85, 90, 95, 96, 97, 98, 99% or more identical (or hybridize under highly stringent hybridization conditions to a complement of a nucleic acid sequence listed in Table A) over a length of at least about 10, 20, 40, 60, 80, 100, 150, 200 or more nucleotides or over the entire length of the reference nucleic acid sequences in Table A. Fragments of the reference nucleic acid sequences in Table A—or similar nucleic acid sequences—can be of any length sufficient to distinguish the fragment from other sequences expected to be present in a mixture, e.g., at least 5, 10, 15, 20, 40, 60, 80, 100, 150, 200 or more nucleotides or at least about 5, 10, 20, 30, 40, 50, 60, 70, 80, 90, 95% of the length of the reference nucleic acid sequences in Table A. “Highly stringent hybridization” means hybridization conditions comprising about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

Levels of protein gene expression products can be measured by any suitable means and in certain embodiments are measured by optical, mechanical, acoustic, thermal or physical methods therefore examples given but not limited to detection with immunoassay, Western blotting, ELISA (enzyme-linked immunosorbent assay), MSIA (mass spectrometric immunoassay), MS/MS (tandem mass spectrometry), RIA (radioimmunoassay), peptide sequencing, flow cytometry, surface plasmon resonance, aptamer-based assay, various multiplexing formats (e.g., LUMINEX® XMAP® technology), QUANTERIX® SIMOA™, bead based detection systems, spectroscopic methods, interferometry, chromatographic methods, colorimetric methods, HPLC, NMR-based technologies; colorimetric, fluorescent, luminescent, magnetic, electrical, piezoelectrical, electrochemical read-out systems with different binders, such as, but not limited to antibodies, antigen-binding fragments of antibodies, sc-Fragments (single-chain fragments), affibodies, nanobodies, aptamers and the like. Protein gene expression products measured in the methods provided by the invention can be of the genes listed in Table A, as well as fragments of these sequences, similar peptide sequences, and fragments of similar peptide sequences. “Similar peptide sequences” can be naturally occurring (e.g., allelic variants or homologous sequences from other species) or engineered variants to the genes in Table A and will exhibit substantially the same biological function and/or will be at least about 60, 65, 70, 75, 80, 85, 90, 95, 96, 97, 98, 99% or more homologous (i.e., conservative substitutions (see, e.g., Heinkoff and Heinkoff, PNAS 89(22):10915-10919 (1992) and Styczynski et al., Nat. Biotech. 26(3):274-275 (BLOSUM, e.g., BLOSUM 45, 62 or 80) or Dayhoff et al., Atlas of protein sequence and structure (volume 5, supplement 3 ed.), Nat. Biomed. Res. Found. pp. 345-358 (PAM, e.g., PAM 30 or 70))) or identical at the amino acid level over a length of at least about 10, 20, 40, 60, 80, 100, 150, 200 or more amino acids or over the entire length of a protein product of the genes in Table A. Fragments of protein products of the genes in Table A—or similar peptide sequences—can be of any length sufficient to distinguish the fragment from other sequences expected to be present in a mixture, e.g., at least 5, 10, 20, 40, 60, 80, 100, 150, 200 or more amino acids or at least about 5, 10, 20, 30, 40, 50, 60, 70, 80, 90, 95% of the length of protein products of the genes in Table A. In particular embodiments, levels of protein gene expression products are measured by an immunoassay, such as a sandwich immunoassay.

An “immunoassay” is an analytical assay that employs binders for detecting an analyte, e.g., a protein marker, such as a cell-surface protein marker. “Binder” encompasses both immunoglobulins (as well as antigen-binding fragments thereof), soluble receptors (including Fc-fusions thereof), and non-immunoglobulin scaffolds that can be adapted and used similarly to immunoglobulins-so-called “antibody-mimetics.” Exemplary antibody mimetics include those based on lectins, fibronectin 3 domains (Fn3 domains; also known as “monobodies”; see, e.g., Koide and Koide, Methods Mol. Biol. 352:95-109 (2007)), Z domains of protein A (also known as “affibodies”; see, e.g., Nygren, FEBS J. 275(11):2668-76 (2008)), gamma-B crystalline or ubiquitin (afflins; see, e.g., Ebersbach et al., J. Mol. Biol. 372(1):172-85 (2007)), lipocalins (anticalins; see, e.g., Skerra, FEBS 275(11):2677-83 (2008)); A domains of membrane receptors (avimers; see, e.g., Silverman et al., Nat. Biotechnol. 23(12):1556-61 (2005)); ankryn repeats (darpins; see, e.g., Stumpp et al., Drug Discov. Today 13(15-16):695-701 (2008)); SH3 domain of Fyn (fynomers; see, e.g., Grabulovski et al., J. Biol. Chem. 282(5):3196-3204 (2007)), and Kunitz type domains (Kunitz domain peptides; see, e.g., Nixon and Wood, Curr. Opin. Drug Discov. Devel. 9(2):261-8 (2006)). In particular embodiments, the antibody is an immunoglobulin. “Immunoglobulin” refers to both full-length immunoglobulins, as well as antigen-binding fragments of immunoglobulins, such as Fab, F(ab′)2, Fv, scFv, Fd, dAb, and other immunoglobulin fragments that retain antigen-binding function. Immunoglobulins will have at least 3 CDRs (complementarity determining regions) in their antigen-binding domain, and, in more particular embodiments, 4, 5, or 6 CDRS, and, in still more particular embodiments, 6 CDRs in an antigen-binding domain. Immunoglobulins for use in the invention include, for example, human, orangutan, mouse, rat, goat, sheep, rabbit, donkey, guinea pig, and chicken antibodies. Immunoglobulins may be polyclonal, monoclonal, monospecific, polyspecific, non-specific, humanized, camelized, single-chain, chimeric, synthetic, recombinant, hybrid, mutated, or CDR-grafted.

Gene expression levels can be measured from samples obtained at any time during a culture, e.g., from inoculation to harvesting. By the advantageous methods provided by the invention, the differentiation state of a culture can be evaluated much more rapidly than existing methods and, in some embodiments, in an automated fashion. In some embodiments, the expression levels are measured within, relative to obtaining the sample, about: 0 hours, 0.5 hours, 1 hour, 2 hours, 3 hours, 4 hours, 5 hours, 6 hours, 12 hours, 18 hours, or 24 hours; or about: 1 day, 2 days, 3 days, 4 days, or 5 days, or more. In certain embodiments, the sample is obtained, for measuring the expression levels, at about: 0, 5, 10, 15, 30, 45, 60, 75, or 90 minutes; or 2, 3, 4, 5, 6, 12, 18, or 24 hours; or 1, 2, 3, 4, 5, 6, 7, 8, 9, 10, 12, 14, 21, or 28 days, of starting the culture.

Expression levels for a given marker or set of markers can be measured more than once; for example, the expression levels can be measured multiple times in a time series, e.g., at least: 2 times, 3 times, 4 times, 5 times, 6 times, 7 times, 8 times, 9 times, or 10 times. A “time series” of gene expression levels is two or more gene expression levels for a given gene expression product taken at different times. In certain embodiments, multiple measurements are made over a period of about: 1 day, 2 days, 3 days, 4 days, 5 days, 6 days, or 7 days, or about: 1 week, 2 weeks, 3, or 4 weeks, or more.

In particular embodiments, expression levels are measured during the process of: harvesting cells (e.g., MSCs) from the culture, making a cell bank (master or working) from the culture, making the final cell product, or a combination thereof.

### Additional Aspects

The methods provided by the invention can include a variety of additional steps. For example, in some embodiments, the methods provided by the invention further comprise the step of: harvesting cells (e.g., MSCs) from the culture, making a cell bank (master or working) from the culture, making the final cell product, or a combination thereof.

In certain embodiments, the methods can further include a step of determining whether the MSCs exhibit other characteristics of MSCs, such as adherence to plastic, 95% of the cells expressing CD105, CD73, and CD90; 2% or fewer of the cells expressing CD45; CD34; CD14 or CD11b; CD79a or CD19; and HLA-DR; and the capability to differentiate along adipocyte, chondroblast, or osteoblast lineages under standard in vitro differentiating conditions. In certain embodiments, the methods entail visualizing the morphology of cells in a sample from the culture, e.g., to classify the differentiation state of the cells by their morphology.

In other embodiments, the methods provided by the invention entail the step of monitoring the expression levels

In particular embodiments, the methods provided by the invention may entail providing a user-readable display of the measured expression levels, such as one or more graphs of the expression levels, such as a time series. In more particular embodiments, the results may include a summary of the differentiation state of the culture.

In a related aspect, the invention also provides kits for performing the methods provided by the invention—e.g., for measuring the level of markers provided by the invention. The kits can be for either singleplex or multiplex detection of the one or more markers provided by the invention. Reagents for the kit can be microarrays, oligonucleotides, or antibodies. In some embodiments, the reagents for detecting the markers provided by the invention, such as antibodies (or other binders), are detectably labeled.

In yet another related aspect, the invention provides computer-implemented methods, a non-transient, computer-readable medium comprising instructions for performing the methods, and systems comprising the medium and a processor for implementing the instructions. For example, the non-transient computer-readable medium can comprise instructions accepting data representing the expression levels of one or more markers provided by the invention and evaluating the expression levels of the one or more markers, where changes in the expression levels, relative to suitable controls, indicate a change in the differentiation state of the culture. The medium can further provide instructions to provide a user-readable display of results of the method, as described elsewhere in the application.

### Methods of Treatment

In another aspect, the invention provides method of treating a subject in need of cells selected from MSCs, adipocytes, chondroblasts, or osteoblasts, by providing the subject a therapeutically effective amount of the cells from a culture tested by the method of any one of the methods provided by the invention and determined to contain cells in the necessary differentiation state.

A “subject” refers to a mammal, including primates (e.g., humans or monkeys), cows, sheep, goats, horses, dogs, cats, rabbits, guinea pigs, rats, mice, or other bovine, ovine, equine, canine, feline, rodent or murine species. Examples of suitable subjects include, but are not limited to, human patients. While subjects may be of any stage of life and any age, e.g., neonate, infant, toddler, child, young adult, adult, or geriatric, in particular embodiments the subject is an adult, e.g., a human adult, i.e., about 18 years old, or older, e.g., about: 18-70 years old, 20-60 years old, 25-55 years old, 25-50 years old, 30-50 years old, or 25-65 years old, as well as greater than about: 30 years old, 40 years old, 50 years old, 60 years old, 70 years old, 80 years old or 90 years old.

As used herein, the terms “treat,” “treating,” or “treatment” mean to counteract a medical condition so that the medical condition is improved according to a clinically acceptable standard.

As used herein, a “therapeutically effective amount” is an amount sufficient to achieve the desired therapeutic or prophylactic effect under the conditions of administration, such as an amount sufficient to treat a given condition. The effectiveness of a therapy can be determined by one skilled in the art using standard measures and routine methods.

### EXEMPLIFICATION

### Methods and Results

The antibodies used in these examples are described in Table B, below. Other antibodies—either commercially available or customized—or binders, as described above, can be used analogously, as described in the application.

TABLE B

| | | Bead | | | Further | |
|---|---|---|---|---|---|---|
| Analyte | Application | #¹ | Vendor | Cat # | Processing | Reactivity |
| KLF4 | Capture | M36 | R&D Systems | AF3640 | Conjugation | H |
| KLF4 | Detection | — | R&D Systems | BAF3640 | None | H |
| KLF5 | Capture | M20 | Novus | NBP1-88508 | buffer exchange | H |
| | | | Biologicals | | and conjugation | |
| KLF5 | Detection | — | EMD Millipore | 07-1580 | buffer exchange | M, R, H |
| | | | | | and biotinylation | |
| PAI-1 | Capture | M19 | Technoclone | TC21173 | buffer exchange | H |
| | | | | | and conjugation | |
| PAI-1 | Detection | — | Technoclone | TC21193 | buffer exchange | H |
| | | | | | and biotinylation | |
| FABP4 | Capture | M66 | R&D Systems | AF3150 | Conjugation | H |
| FABP4 | Detection | — | R&D Systems | AF3150 | Biotinylation | H |
| Chemerin | Capture | M74 | R&D Systems | MAB23241 | Conjugation | H |
| Chemerin | Detection | — | R&D Systems | BAF2324 | None | H |
| ADIPOQ | Capture | M51 | Therapeutic | AD72 | Conjugation | H |
| ADIPOQ | Detection | — | Labs, | AD105 | Biotinylation | H |
| | | | Alan Epstein | | | |
| Runx2 | Capture | M30 | R&D Systems | AF2006 | Conjugation | H |
| Runx2 | Detection | — | R&D Systems | BAF2006 | None | H |
| ALPL | Capture | M53 | EMD Millipore | MAB4349 | buffer exchange | H, Po, Rb, |
| | | | | | and conjugation | Fe |
| ALPL | Detection | — | EMD Millipore | MAB4354 | buffer exchange | H, Mk, |
| | | | | | and biotinylation | Bab, Chp, |
| | | | | | | Pm |
| Osteonectin | Capture | M54 | Meridian Life | H95031M | Conjugation | H, R |
| | | | Science | | | |
| Osteonectin | Detection | — | R&D Systems | MAB941 | Biotinylation | H |
| Osteocalcin | Capture | M63 | Serotec | 0400-0042 | buffer exchange | H, Rb, R, |
| | | | | | and conjugation | Po |
| Osteocalcin | Detection | — | Serotec | 0400-0039 | buffer exchange | H |
| | | | | | and biotinylation | |
| CTNNB1 | Capture | M22 | BD Biosciences | 610154 (624084) | buffer exchange | H, M, R, |
| | | | | | and conjugation | Do, Ch |
| CTNNB1 | Detection | — | Invitrogen | AHO0462 | buffer exchange | H, M, Ra |
| | | | | | and biotinylation | |
| Sox9 | Capture | M14 | Millpore | AB5535 | buffer exchange | H, R, M |
| | | | | | and conjugation | |
| Sox9 | Detection | — | R&D Systems | BAF3075 | None | H |
| MIA | Capture | M34 | R&D Systems | AF2050 | Conjugation | H |
| MIA | Detection | — | R&D Systems | BAF2050 | None | H |
| COMP | Capture | M18 | BioVendor | RD182080100F1 | Conjugation | H |
| COMP | Detection | — | LS Bio | LS-C122806 | Biotinylation | H |
| COL2A1 | Capture | M42 | Abnova | H00001280-M06 | Conjugation | H |
| COL2A1 | Detection | — | Abnova | H00001280-M01 | Biotinylation | H |

¹bead region (plus M for magnetic)

### Differentiation Controls

hMSCs are functionally defined by their capacity to self-renew and their ability to differentiate into multiple cell types, including adipocytes, chondrocytes, and osteocytes. These protocols describe the differentiation of hMSCs into three lineages. Cells were expanded in T150 flasks and differentiation assays were conducted for 21 days. Supernatants and lysates were collected at different intervals by harvesting a designated flask on a particular day. On completion of differentiation procedure, immunostaining was performed to confirm the lineage.

### Adipogenic Differentiation

hMSCs were scaled up in T150 flasks in DMEM (1 g/L glucose, INVITROGEN®) supplemented with 10% fetal bovine serum, 2 mM L-Glutamine, 100 U/ml penicillin, 0.1 mg/ml streptomycin, and 8 ng/ml basic fibroblast growth factor (EMD MILLIPORE). After expansion of cells, adipogenesis was induced using INVITROGEN® MEM Alpha supplemented with 10% fetal bovine serum, 2 mM L-glutamine, 100 U/ml penicillin, 0.1 mg/ml streptomycin and 1× StemXVivo Adipogenic supplement (R&D SYSTEMS®). At the start of adipogenesis, initial seeding density of the cells was 2.1E4 cells/cm². Cells were maintained in parallel in flasks with and without supplement. Media change was performed twice a week on flasks with and without supplements. One flask with regular growth media was maintained by passaging cells twice a week for 21 days. Supernatants and lysates were collected at different time intervals. Lysates were generated using EMD MILLIPORE's MILLIPLEX® MAP lysis buffer supplemented with Protease Inhibitor Cocktail Set VII and BENZONASE® Nuclease.

Oil Red Staining:

At the end of 21 days, adipocytes were fixed with 4% paraformaldehyde for 30-40 min at room temperature. Cells were washed with PBS and water three times. Cells were stained with Oil Red solution 0.36% in 60% isopropanol (EMD MILLIPORE) for 50 min. Cells were then washed with water and the stained fat droplets were visualized by microscopy and photographed. The presence of stained oil droplets indicates the cells have undergone adipogenic differentiation.

### Osteogenic Differentiation

hMSCs were scaled up in T150 flasks in DMEM (low glucose, INVITROGEN®) supplemented with 10% fetal bovine serum, 2 mM L-glutamine, 100 U/ml penicillin, 0.1 mg/ml streptomycin, and 8 ng/ml basic FGF. After expansion of cells, osteogenesis was induced using INVITROGEN® MEM Alpha supplemented with 10% fetal bovine serum, 2 mM L-Glutamine, 100 U/ml penicillin, 0.1 mg/ml streptomycin, and R&D SYSTEMS® StemXVivo Osteogenic supplement (1×). At the start of osteogenesis, initial seeding density for the cells was 4.2E3 cells/cm². Cells were maintained in parallel in flasks with and without supplement. Media change was performed twice a week on flasks with and without supplements. One flask with regular growth media was maintained by passaging cells twice a week for 21 days. Supernatants and lysates were collected at different time intervals. Lysates were generated using EMD MILLIPORE's MILLIPLEX® MAP lysis buffer supplemented with Protease Inhibitor Cocktail Set VII and BENZONASE® Nuclease.

Alizarin Red Staining:

At the end of 21 days, Osteocytes were fixed with 70% ethanol for 1 hour at room temperature. Cells were washed with water three times and stained with Alizarin Red (EMD MILLIPORE) for 30 min. Cells were then washed with water and the mineralized nodules were visualized by microscopy and photographed. The presence of stained calcium deposits indicates the cells have undergone osteogenic differentiation.

### Chondrogenic Differentiation

hMSCs were seeded at 2E5 cells/well in BD Falcon round bottom 96 well plates. Cells were spun down and Chondrogenesis was initiated using Gibco Stem Pro Chondrogenesis differentiation kit which includes media and supplement. Five plates were maintained with supplements and five were without. One plate was maintained with regular MSC growth media control for the entire duration of 21 days. All the 96 well plates were run in parallel and media change was performed twice a week. Two plates with and without supplement were harvested at different time intervals. Spheroid generation in supplemented plates was observed after 5-6 days. At the time of harvest, plates were spun at 2000 RPM for five minutes and spheroids in all the wells were washed with tris-buffered saline supplemented with Protease Inhibitor Cocktail Set VII (EMD MILLIPORE). The buffer was removed with multichannel pipette and plates were stored in −80° C. After all the samples were collected at various time intervals, spheroids were lysed using EMD MILLIPORE's MILLIPLEX® MAP lysis buffer supplemented with Protease Inhibitor Cocktail Set VII and BENZONASE® Nuclease. Covaris Ultrasonicator S220 was used to lyse the samples.

Chondrogenesis Spheroids for Sectioning and Alcian Blue Staining:

21 day old spheroids were fixed with 4% paraformaldehyde for 30 min and treated with 20% glucose solution in water (FLUKA®) 5-10 min. A tissue embedding mold was filled with OCT embedding compound (Electron Microscopy Sciences). Molds were kept on dry ice and spheroids were transferred using an inoculation loop (BD) onto the semi frozen OCT. An additional drop of OCT was added to further embed the spheroids in the molds. Spheroid sections were generated using a cryostat. Sections were placed on slides. An aqua hold barrier pap pen (Scientific device laboratory) was used to circle the sections on the slide. Sections were stained with 1% Alcian blue solution (Electron Microscopy Sciences) for 30 minutes at room temperature and then washed with water three times and visualized by microscopy and photographed. The presence of stained proteoglycans indicated that the cells underwent chondrogenic differentiation.

### Assay Development for Three Lineages

### Material

Buffers and Solutions:
MILLIPLEX® MAP Lysis Buffer (EMD MILLIPORE, Catalogue No. 43-040)
MILLIPLEX® MAP Assay Buffer 1 (EMD MILLIPORE, Catalogue No. 43-010)
MILLIPLEX® MAP Streptavidin-Phycoerythrin (Cat. #45-001H)
MILLIPLEX® MAP Amplification buffer (EMD MILLIPORE, Catalogue No. 43-024A)
Protease Inhibitor Cocktail Set VII (EMD MILLIPORE, Catalogue No. 539138-1SET)
Microtiter filter-plates: MULTISCREEN HTS-BV (EMD MILLIPORE, Catalogue No. MSBVN1210)
Vacuum module for filter-plates: MULTISCREEN® Vacuum Manifold 96-well (EMD MILLIPORE, Catalogue No. MAVM0960R)
LUMINEX® 200™ system (EMD MILLIPORE, Catalogue No. 40-013)

### Methods

### Cell Lysate Preparation

Cells were washed with ice-cold phosphate-buffered saline. MILLIPLEX® MAP Cell Signaling Lysis Buffer with freshly added protease inhibitors was added to cells (1 mL per 1×107 cells). The suspension was rocked for 10-15 min at 4° C. The lysates were then filtered through a 0.1-μm membrane filter and total protein was quantified using a BCA assay.

### Development of Bead-Based Immunoassays

Bead-based immunoassays were developed by conjugating specific capture antibodies to MagPlex® microsphere beads purchased from Luminex Corp. These antibodies are declared as “Capture” Table B. Each set of beads is distinguished by different ratios of two internal dyes, yielding a unique fluorescent signature to each bead set indicated by the bead number. Analyte binding to these capture beads was subsequently detected by a secondary biotinylated detection antibody, declared as “Detection” Table B, in a reaction with MILLIPLEX® MAP Streptavidin-Phycoerythrin.

### MILLIPLEX® MAP Assay Protocol

Preparation of Lysate Samples:

The cell lysates were adjusted to a final total protein concentrated of 400 μg/mL by adding an appropriate volume of MILLIPLEX® MAP Assay Buffer 1. They were kept on ice while preparing the microtiter plates for the assay.

Assay Procedure:

The multiplex assay was performed in a 96-well filter plate. Each well of the plate was first filled with 50 μL MILLIPLEX® MAP Assay Buffer 1 and incubated on a plate shaker (600-800 rpm) for 10 minutes at room temperature (20-25° C.).

The MILLIPLEX® MAP Assay Buffer 1 was removed from all wells by vacuum filtration. The filter plate was placed on the vacuum module, vacuum was applied to remove the liquid and the filter plate was gently blotted on the bottom on a paper towel to remove excess liquid. The respective suspension of capture beads containing 120000 beads/mL was vortexed for 10 seconds and 25 μL of this bead suspension was added to each of the respective wells. Controls, cell lysates (10 μg total protein) or cell culture supernatants were diluted 1:1 in MILLIPLEX® MAP Assay Buffer 1, resulting in a total volume of 25 μL. These samples were added to the appropriate wells and the plate was incubated overnight (16-20 hours) at 2-8° C. on a plate shaker (600-800 rpm) protected from light. The samples were removed by vacuum filtration as described above. 100 μL MILLIPLEX® MAP Assay Buffer 1 was added to each well. The plate was shaken for 30 seconds on a plate shaker (600-800 rpm) and the liquid was removed from all wells by vacuum filtration as described above. This procedure was repeated for a total of two washes. 25 μL of 1× detection antibody solution was added to each well, and the plate was sealed with a lid and incubated with agitation on a plate shaker (600-800 rpm) for 1 hour at room temperature (20-25° C.). The liquid was removed from all wells by vacuum filtration as described above. 25 μL MILLIPLEX® MAP streptavidin-phycoerythrin (SAPE) was added to the wells, and the plate was sealed with a lid and incubated with agitation on a plate shaker (600-800 rpm) for 15 minutes at room temperature (20-25° C.). An additional 25 μL of MILLIPLEX® MAP Amplification Buffer was added to each well, and the plate was sealed with a lid and incubated with agitation on a plate shaker (600-800 rpm) for 15 minutes at room temperature (20-25° C.). The liquid was removed from all wells by vacuum filtration as described above. The beads were suspended in 150 μL MILLIPLEX® MAP Assay Buffer 1 and mixed on plate shaker (600-800 rpm) for 5 minutes at room temperature (20-25° C.) and subsequently analyzed using a LUMINEX® 200™ system.

### Results

### Exemplary Results for Differentiation Monitoring

Exemplary results for the monitoring of hMSC differentiation with the assays disclosed in the application were generated using the LUMINEX® technology. The output of a LUMINEX® measurement is given in Median Fluorescence Intensity (MFI) that is correlated with the concentration of the target analyte/protein. Thus monitoring of the MFI throughout the course of a cultivation or differentiation experiment gives a qualitative time-discrete picture of the respective target protein expression.

Since hMSCs have the ability to differentiate in the three different lineages of adipocytes, osteoblasts and chondrocytes, three different MILLIPLEX® MAP panels are disclosed in this invention. Subsequently, exemplary results for differentiation experiments of hMSC in all three lineages will be shown that demonstrate the applicability of the described invention.

The differentiation of hMSCs into the adipocyte lineage took 21 days and could be monitored via the markers KLF4, KLF5, PAI-1, FABP4, Chemerin and Adiponectin (ADIPOQ). To show the time-discrete monitoring ability during a course of differentiation, samples were taken on days 0, 1, and 6 as early time-points, at day 12 as an intermediate time-point and at day 21 as a late time-point. For analysis, the cell samples were lysed and applied to the MILLIPLEX® MAP Assay Protocol for all six markers.

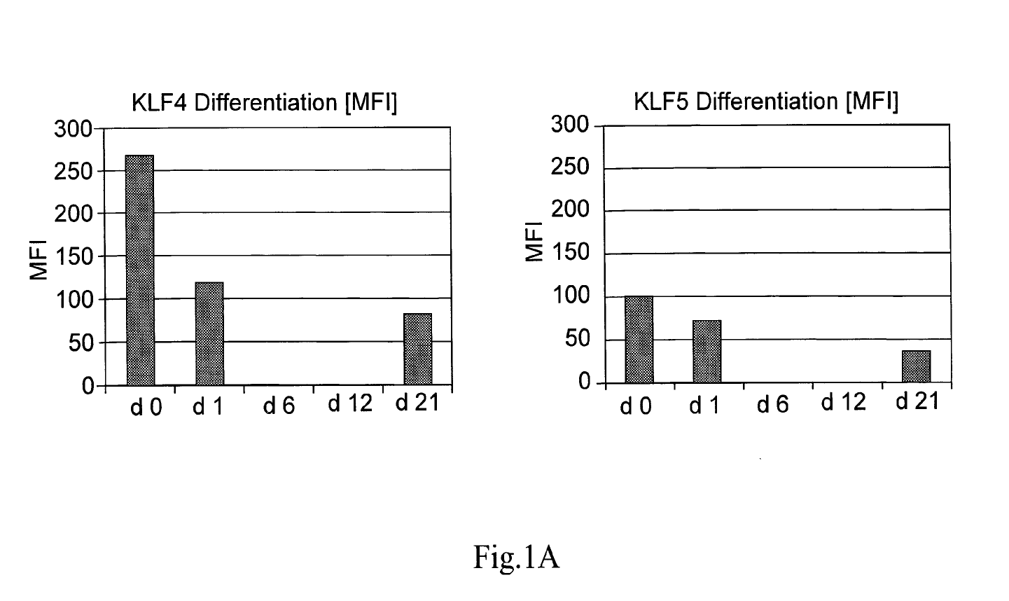

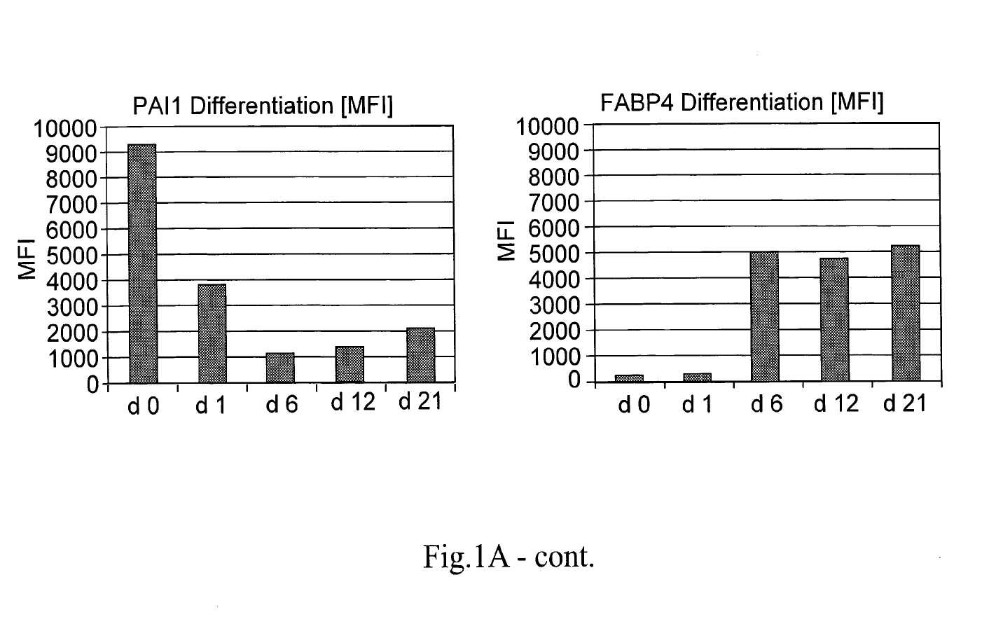

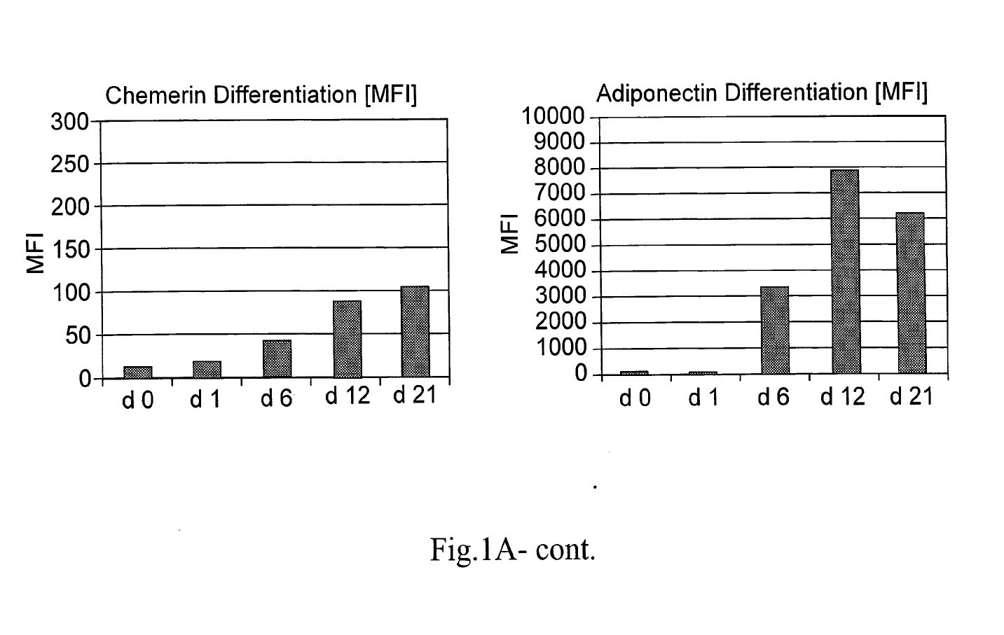

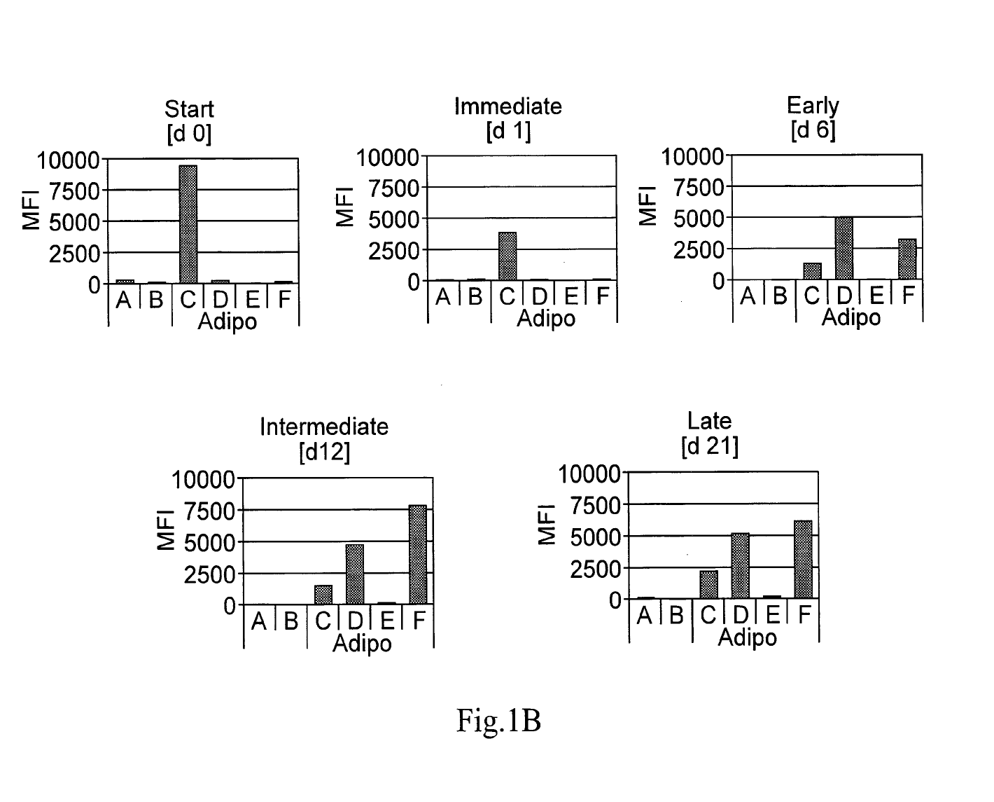

For the adipocyte lineage, the chosen differentiation markers exhibited a time-dependent expression behavior. The early markers KLF4, KLF5, and PAI-1 were expressed in the early days of the differentiation and the relative protein levels declined over differentiation-time. The intermediate and late markers FABP4, Chemerin and Adiponectin (ADIPOQ) showed an onset of expression on day six with a clear increase until the end of the differentiation experiment (day 21). Thus, these markers could be applied for monitoring the changes in protein expression representing the differentiation state of the hMSCs (FIG. 1A). The differential expression profile of the analyzed markers became even more apparent when shown in the same bar graph (FIG. 1B).

As the second lineage, the differentiation into osteoblasts was subject to differentiation monitoring by the disclosed MILLIPLEX® MAP assays. Osteogenesis could be monitored using the markers Runx2, Alkaline Phosphatase (ALPL), Osteonectin, Osteocalcin and Beta-Catenin (CTNNB1). For this differentiation, the cells were again processed for 21 days as described in the protocol section. The samples were taken at days 0, 1, and 6 for observing the early expressed markers, on day 12 to cover intermediate expression and on day 21 for monitoring late expression. The expression of Beta-Catenin, Runx2, ALPL and Osteocalcin was analyzed using cell lysates as described in the MILLIPLEX® MAP Assay Protocol. Only for measuring the expression of Osteonectin cell culture supernatants were used.

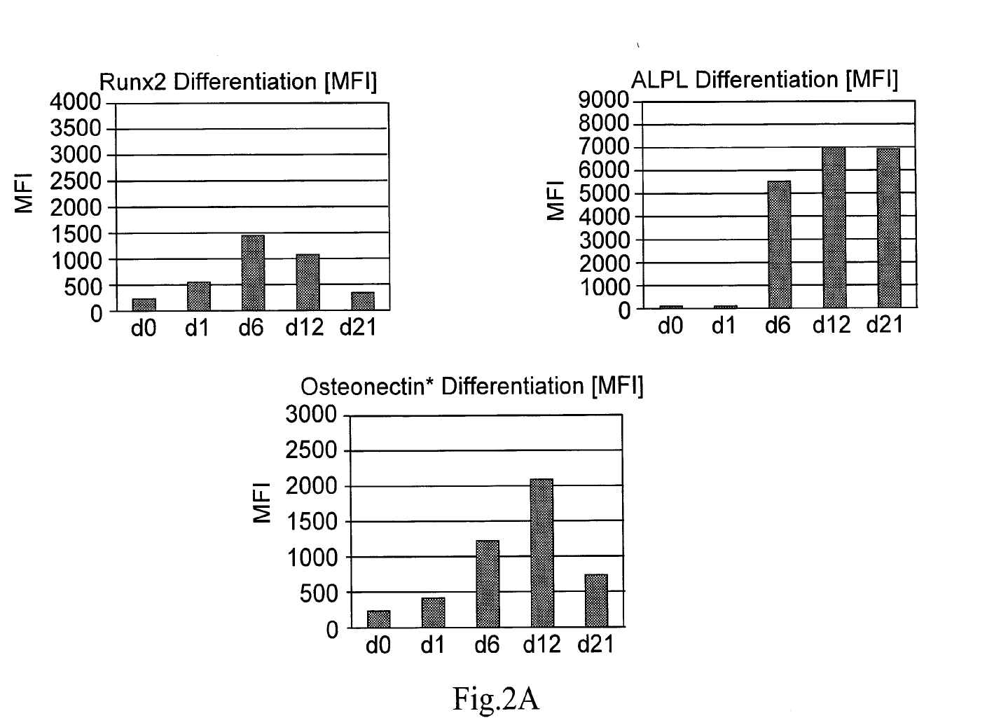

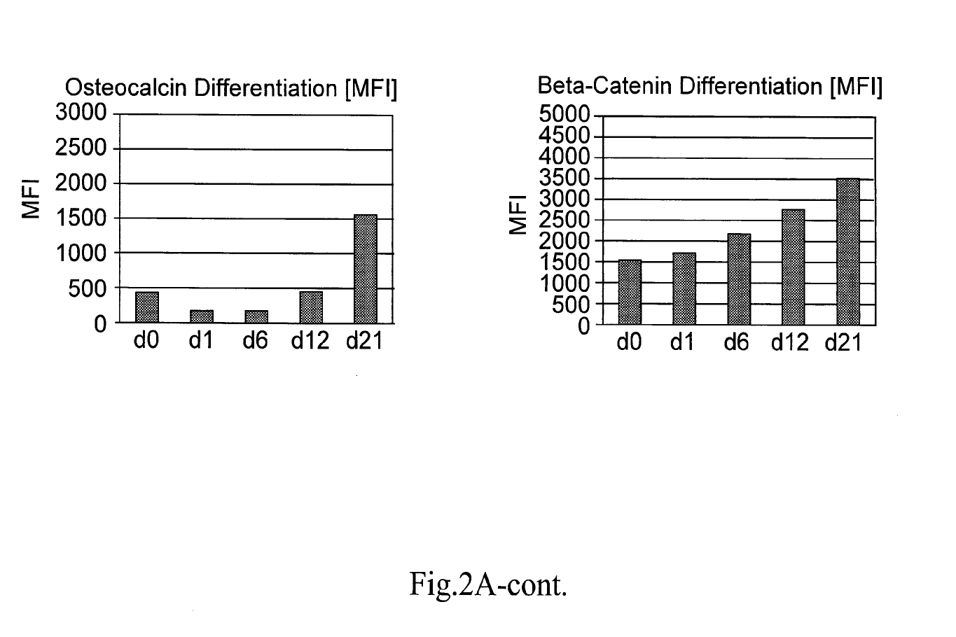

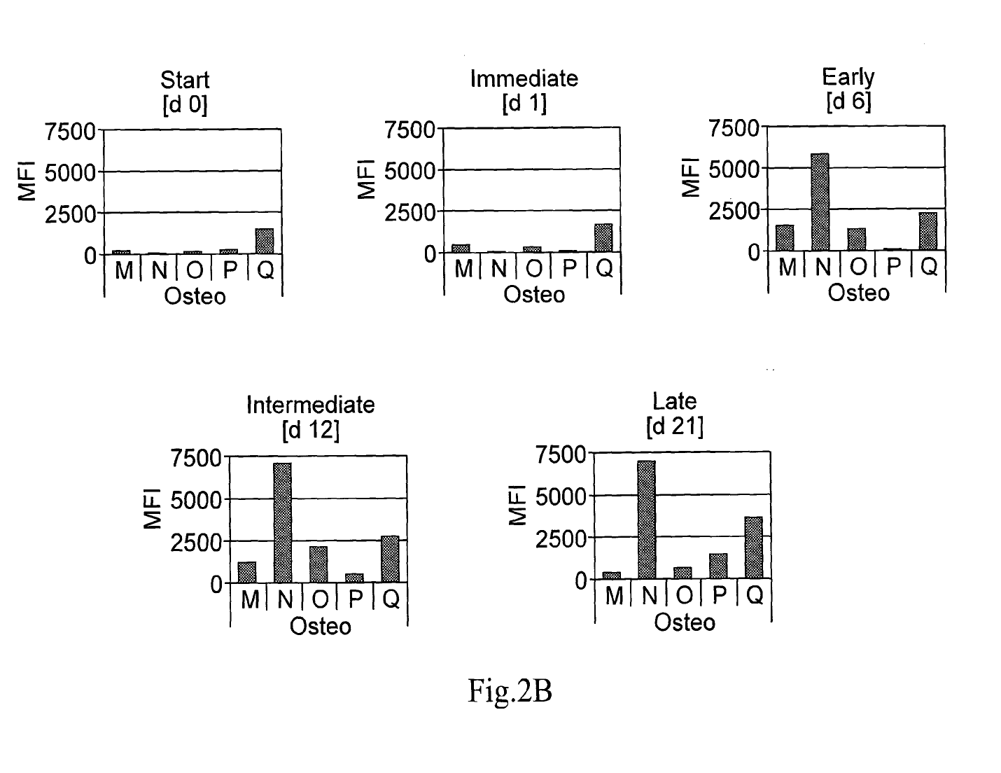

For the osteoblast lineage the selected protein markers exhibited time dependent expression patterns. For Runx2 an early/intermediate expression was observed with a peak expression around day 6. ALPL expression started in the intermediate differentiation time and stayed constant over the subsequent time-points. For Osteonectin a clear peak of secretion was observed in the intermediate timeframe. In contrast to this Osteocalcin exhibited a clear late onset of expression. The level of Beta-Catenin was high throughout the entire differentiation process with an increase towards the endpoint on day 21 (FIG. 2A). The applicability of the 5 described markers for monitoring the osteoblast differentiation states is confirmed by data aggregation in the fingerprint view (FIG. 2B).

The third cell type hMSC's can differentiate in is cartilage by going through the chondrocyte lineage. The chondrogenesis markers that were monitored by the disclosed MILLIPLEX® MAP assays are SOX9, MIA, COMP and COL2A1. For this analysis samples from the differentiation procedure were taken on day 0 and 6 for early expression onset on day 14 as intermediate time point and on day 21 for a late induction of protein expression. All four markers were analyzed by using cell lysates as described in the MILLIPLEX® MAP Assay Protocol.

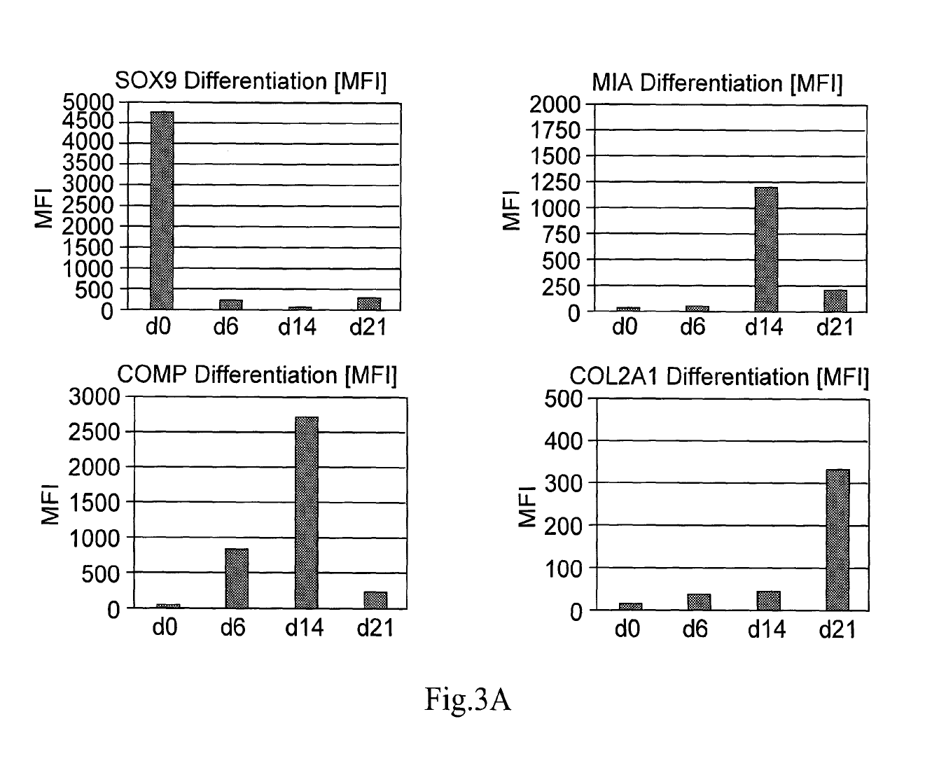

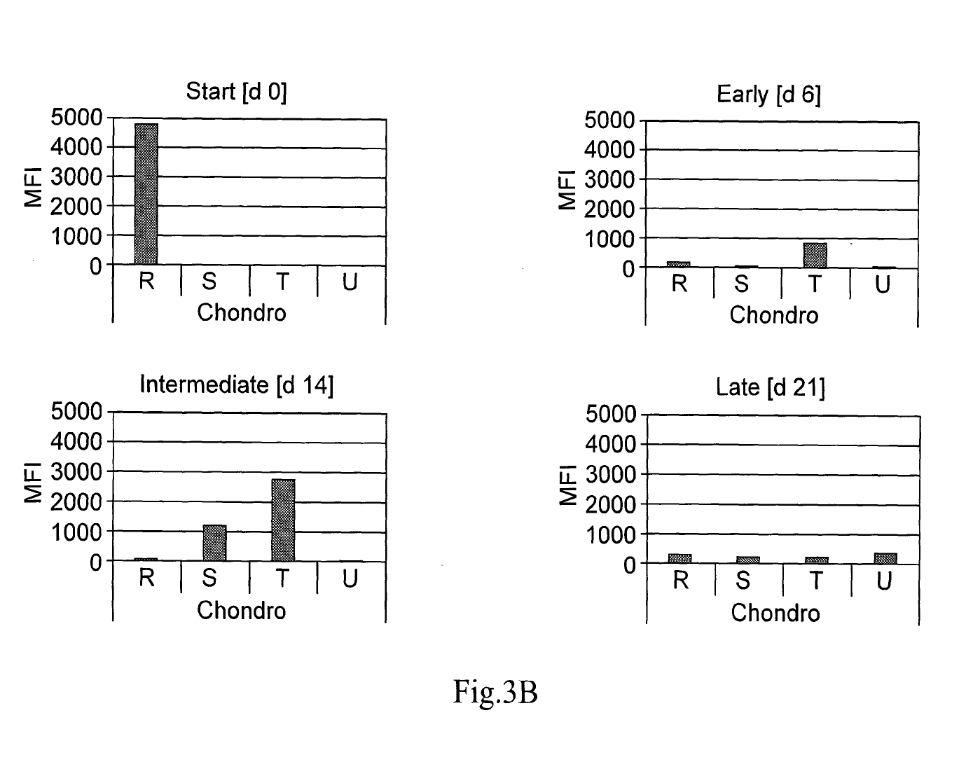

For the chondrogenesis lineage markers, a time dependent expression pattern could be observed. SOX9 was applied as an immediate marker since it is already expressed at the starting point of the differentiation, whereas COMP showed an early intermediate expression that decreased towards the end of the differentiation procedure. MIA served as an intermediate marker since it was exclusively expressed around day 14. The chondrogenesis panel was completed by COL2A1, which was expressed towards the last day of the procedure (FIGS. 3A-3B).

### SUMMARY

These results showed that the selected marker panels allowed a monitoring of hMSC differentiation into all three possible lineages by representing the discrete expression profiles with the measured MFI values. Hence, the differentiation, as well as the undifferentiated state of MSCs, was observed.

### Bioreactor Testing

### Sampling Bioreactor

Sampling of the reactor was performed through a modified sample port that accommodated sampling cells on microcarriers. The sample port was sprayed with 70% ethanol and allowed to air dry for few seconds. A Luer-Lok™ syringe of appropriate size was used, and the syringe was attached into the sample port and turned clockwise to lock the syringe in the sample port. Either 2.4 ml or 4.8 ml was withdrawn. The syringe was slowly turned counterclockwise and removed from the reactor. The sample was emptied into a 15 ml tube and the syringe discarded. Immediately the sample port was sprayed with 70% ethanol and allowed to air dry.

### Cell Lysate Preparation

The cell-microcarrier pellet was washed with ice-cold phosphate-buffered saline with protease inhibitors. MILLIPLEX® MAP Cell Signaling Lysis Buffer (1×, EMD MILLIPORE) with freshly added protease inhibitors and BENZONASE® was added to the cell pellet (400 μL per sample). The suspension was incubated with rocking for 20 min at 4° C. The lysates were then filtered through a 100 micron mesh filter and total protein was quantified using a BCA assay.

### Bead-Based Assay

The bead based assays were used as described in the MILLIPLEX® MAP Assay Protocol, above. Briefly, lysate samples were adjusted to a protein concentration of 200 or 400 μg total protein/mL and assayed in 96-well plates at a concentration of 10 μg total protein/well.

### Results

When expanding hMSCs to large numbers using bioreactors, one must confirm that the cells remain in an undifferentiated state. A subset of the developed assays were used to measure specific target proteins on a daily/regular basis from bioreactors. Samples were withdrawn from the vessel, lysed, filtered to retain microcarriers and the resulting protein material was used in the bead based immunoassay.

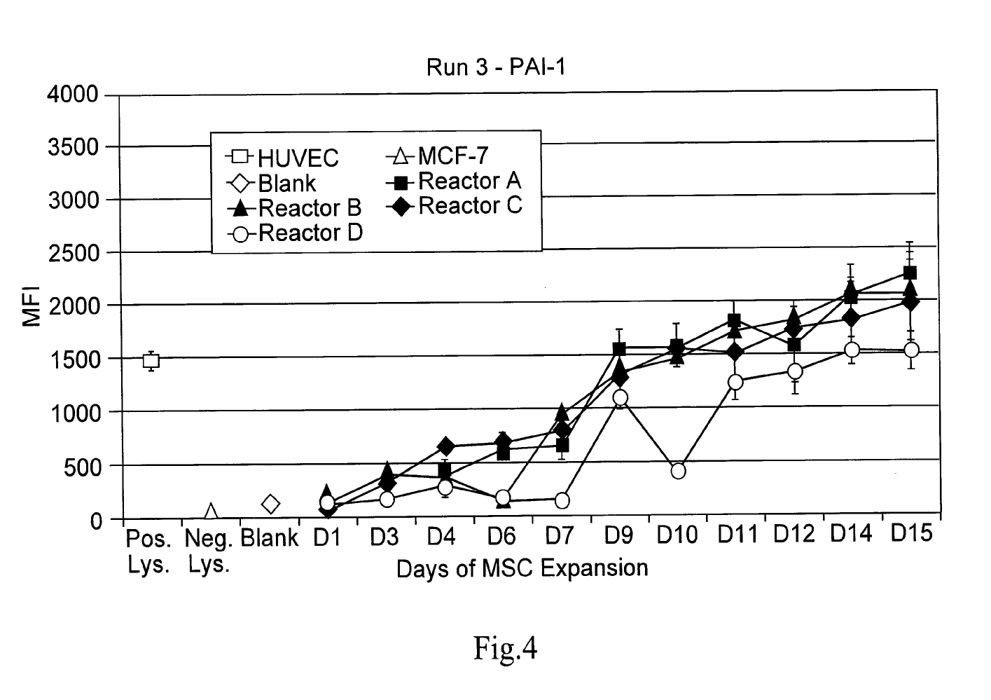

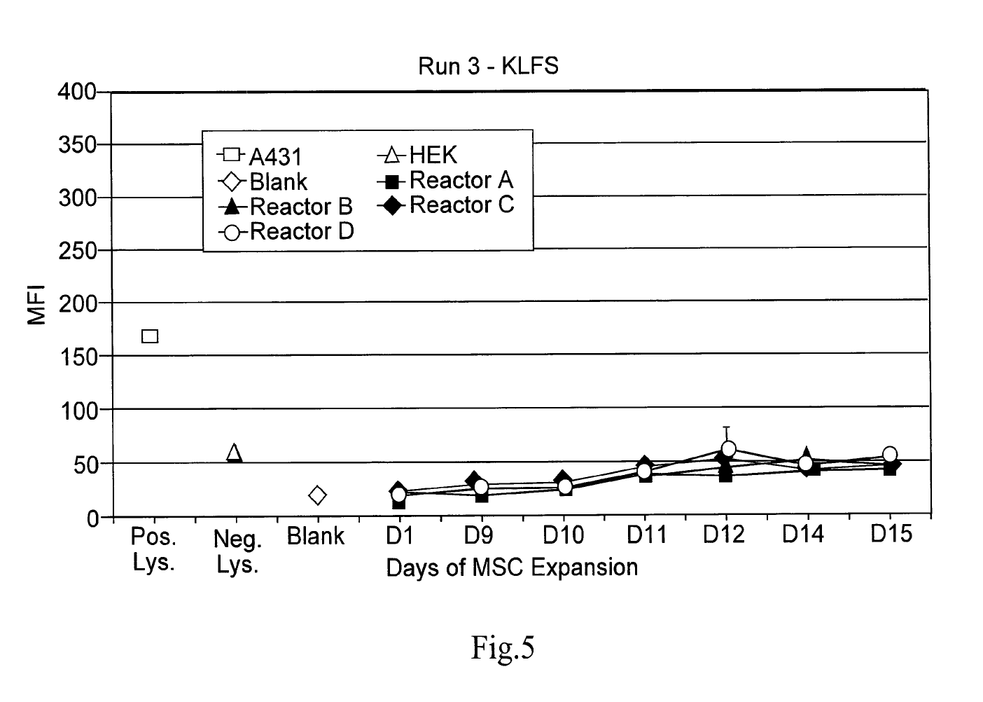

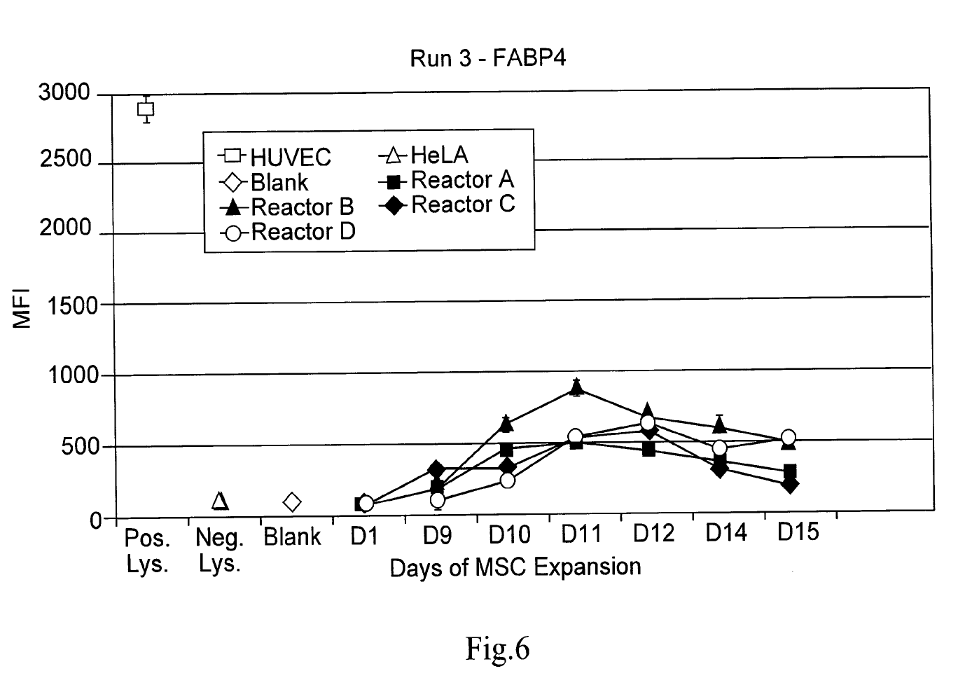

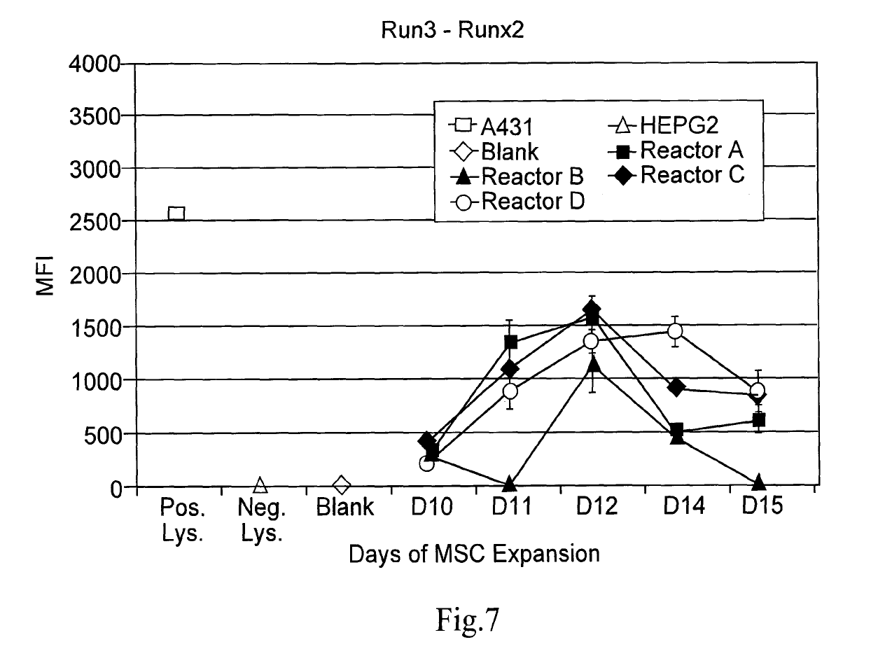

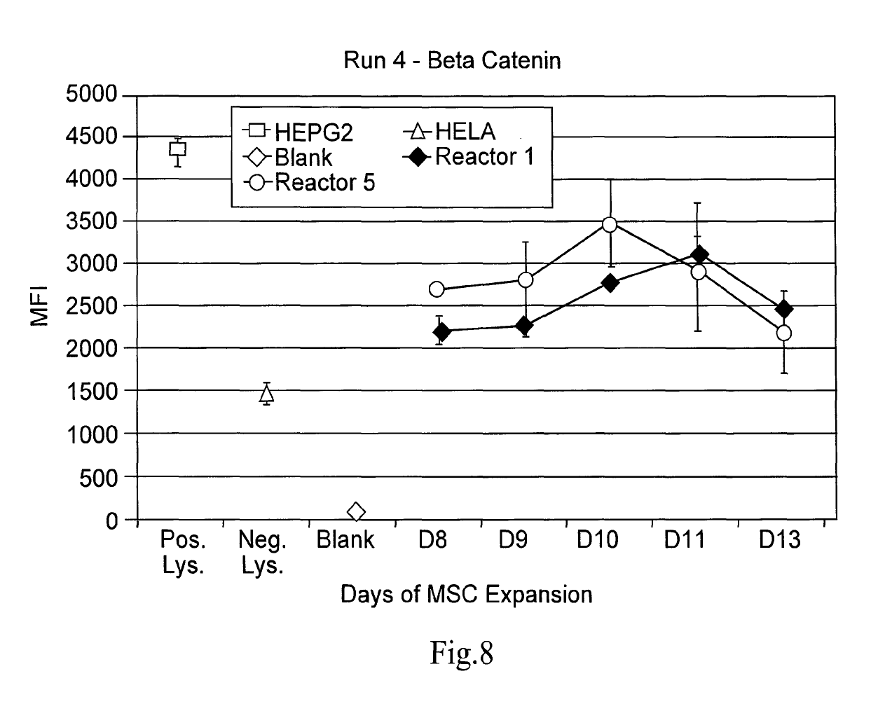

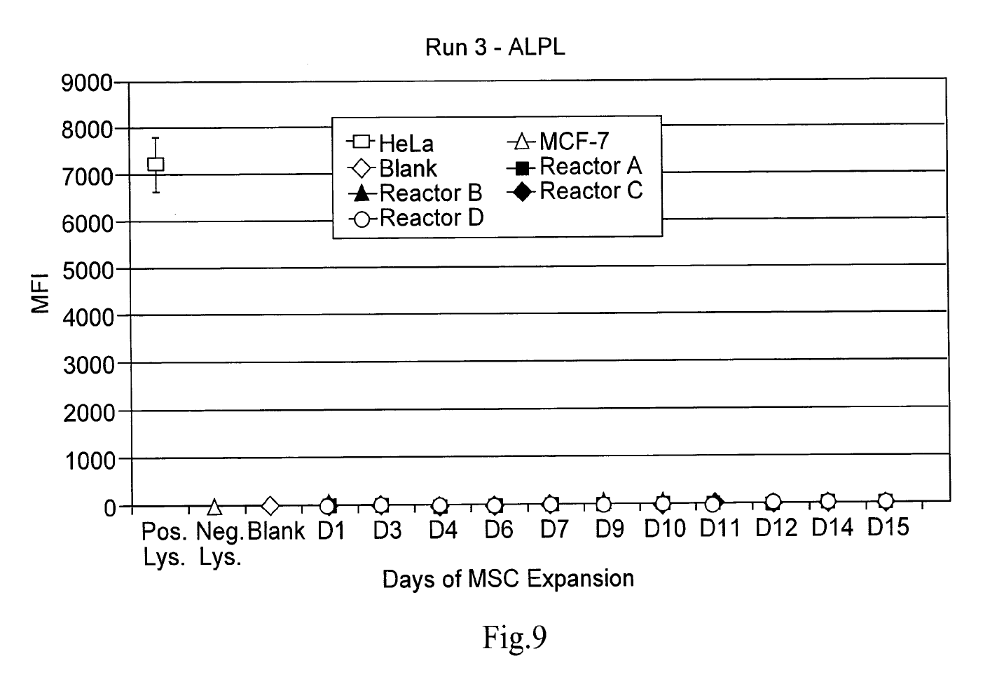

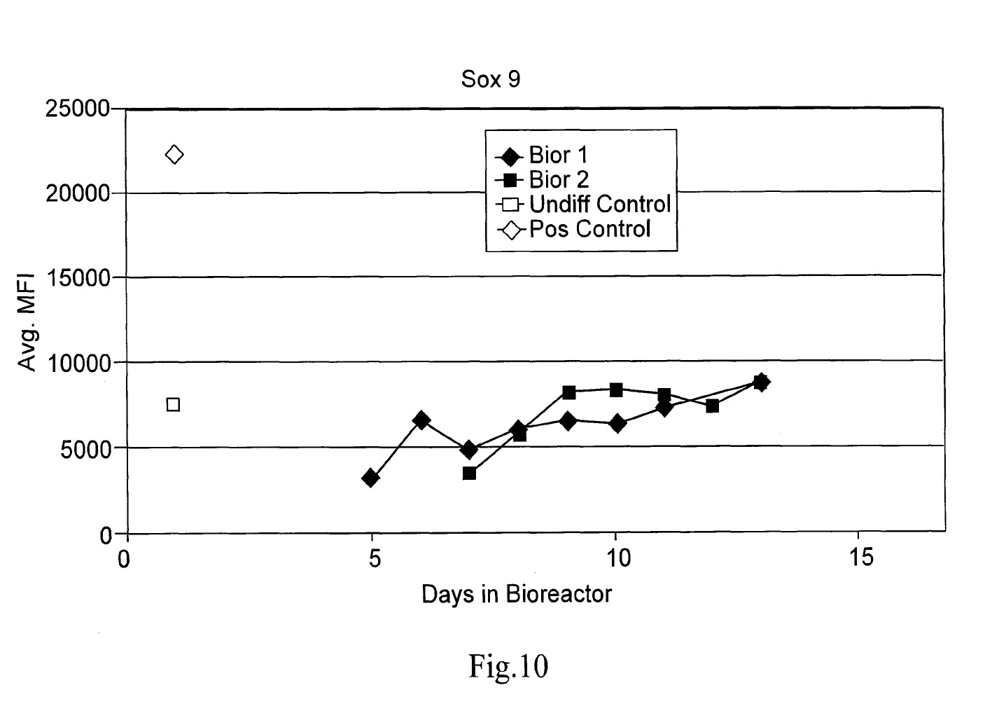

For adipogenesis, Pai-1, Klf5 and FABP4 were assessed. Results are shown in FIGS. 4-6. For osteogenesis, Runx2, beta-catenin and ALPL were assessed. Results are shown in FIGS. 7-9. Overall, markers of adipogenesis and osteogenesis were expressed in cells expanded in bioreactors at similar levels and patterns as observed in undifferentiated and differentiated hMSCs. For chondrogenesis, Sox9 was assessed. The results are shown in FIG. 10.

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., Pub Chem compound, Pub Chem substance, or Pub Chem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the invention are applicable to all of the other aspects of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each various individual and collective combination and permutation of these compounds may not be explicitly disclosed, each is specifically contemplated and described herein. Thus, if a class of elements A, B, and C are disclosed as well as a class of elements D, E, and F and an example of a combination of elements A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed invention by, inter alia, a negative proviso or disclaimer of the feature or combination of features.

The described computer-readable implementations may be implemented in software, hardware, or a combination of hardware and software. Examples of hardware include computing or processing systems, such as personal computers, servers, laptops, mainframes, and micro-processors. In addition, one of ordinary skill in the art will appreciate that the records and fields shown in the figures may have additional or fewer fields, and may arrange fields differently than the figures illustrate. Any of the computer-readable implementations provided by the invention may, optionally, further comprise a step of providing a visual output to a user, such as a visual representation of, for example, sequencing results, e.g., to a physician, optionally including suitable diagnostic summary and/or treatment options or recommendations.

While this invention has been particularly shown and described with references to example embodiments thereof, it will be understood by those skilled in the art that various changes in form and details may be made therein without departing from the scope of the invention encompassed by the appended claims.

What is claimed is:

1-45. (canceled)

46. A method of monitoring the differentiation state of a mesenchymal stem cell (MSC) culture, comprising measuring the expression level of: one or more markers of adipogenesis, one or more markers of osteogenesis, or one or more markers of chondrogenesis, in an isolated sample from the culture, wherein a change in the expression level of one or more of the markers, relative to a suitable control, indicates a change in the differentiation state of the MSC culture.

47. The method of claim 46, wherein:
the one or more markers of adipogenesis is selected from KLF5, FABP4, Adiponectin/ADIPOQ, Chemerin/RARRES2, KLF4, and PAI1;
the one or more markers of osteogenesis is selected from Runx2, ALPL, Osteocalcin/BGLAP, CTNNB1, and Osteonectin/SPARC; or
the one or more markers of chondrogenesis is selected from Sox9, COL2A1, MIA, and COMP.

48. The method of claim 46, wherein the one or more markers of adipogenesis are selected from KLF5, FABP4, and PAI1.

49. The method of claim 46, wherein the expression levels of the following markers is measured:
(i) two or all three of KLF5, FABP4, and PAI1;
(ii) one or more of Adiponectin/ADIPOQ, Chemerin/RARRES2, and KLF4 are measured in addition to KLF5, FABP4, or PAI1; or
(iii) KLF5, FABP4, Adiponectin/ADIPOQ, Chemerin/RARRES2, KLF4, and PAI1.

50. The method of claim 46, wherein the expression levels of the following markers of osteogenesis is measured:
(i) two or all three of Runx2, ALPL, and CTNNB1;
(ii) one or more of Osteocalcin/BGLAP and Osteonectin/SPARC in addition to Runx2, ALPL, and CTNNB1; or
(iii) Runx2, ALPL, Osteocalcin/BGLAP, CTNNB1, and Osteonectin/SPARC.

51. The method of claim 46, wherein the expression levels of two, three, or all four of Sox9, COL2A1, MIA, and COMP are measured.

52. The method of claim 46, wherein the expression levels the following markers are measured:
(i) KLF5, FABP4, PAI1, Runx2, ALPL, and CTNNB1;
(ii) KLF5, FABP4, PAI1, Runx2, ALPL, CTNNB1, Sox9, COL2A1, MIA, and COMP; or
(iii) KLF5, FABP4, Adiponectin/ADIPOQ, Chemerin/RARRES2, KLF4, PAI1, Runx2, ALPL, Osteocalcin/BGLAP, CTNNB1, Osteonectin/SPARC, Sox9, COL2A1, MIA, and COMP.

53. The method of claim 46, wherein the monitoring of the differentiation state of the MSC culture provides a quality control assay and optionally further comprises: (i) monitoring and/or performing the step of harvesting MSCs from the culture; (ii) monitoring and/or performing the step of making a cell bank from the MSC culture; or (iii) or monitoring and/or making the final cell product.

54. The method of claim 46, wherein the MSC culture is induced to differentiate along a lineage selected from adipocyte, chondroblast, or osteoblast.

55. The method of claim 46, wherein the expression levels of the one or more markers are measured simultaneously.

56. The method of claim 46, wherein the expression levels of the one or more markers are measured sequentially.

57. The method of claim 46, wherein the expression levels are measured at the protein level.

58. The method of claim 57, wherein the protein expression levels are measured by optical, mechanical, acoustic, thermal or physical methods; an immunoassay, Western blotting, ELISA (enzyme-linked immunosorbent assay), MSIA (mass spectrometric immunoassay), MS/MS (tandem mass spectrometry), RIA (radioimmunoassay), peptide sequencing, flow cytometry, surface plasmon resonance, aptamer-based assay, multiplexing, bead based detection systems, spectroscopic methods, interferometry, chromatographic methods, fluorescent methods, colorimetric methods, luminescent methods, magnetic methods, electrical methods, piezoelectrical methods, electrochemical read-out systems, HPLC, or NMR-based technologies.

59. The method of claim 57, wherein the expression levels are measured using an immunoassay.

60. The method of claim 58, wherein the immunoassay is a sandwich immunoassay using, for each marker, a pair of detectably labeled binders that specifically bind the marker.

61. The method of claim 60, wherein the binders are fluorescently labeled and the expression levels are measured by flow cytometry.

62. The method of claim 57, wherein the protein expression levels are measured from the culture supernatant.

63. The method of claim 46, wherein the expression levels are measured at the nucleic acid level.

64. The method of claim 63, wherein the nucleic acid expression levels are measured by quantitative polymerase chain reaction (qPCR), quantitative real-time polymerase chain reaction (qRTPCR), digital droplet PCR, (ddPCR), SAGE (serial analysis of gene expression), sequencing, northern blotting, microarrays, transcription mediated amplification, isothermal amplification, or Southern blotting.

65. The method of claim 46, wherein the expression levels are measured within, relative to obtaining the sample, about: 0 hours, 0.5 hours, 1 hour, 2 hours, 3 hours, 4 hours, 5 hours, 6 hours, 12 hours, 18 hours, or 24 hours; or about: 1 day, 2 days, 3 days, 4 days, or 5 days.

66. The method of claim 46, wherein the sample is obtained, for measuring the expression levels, at about: 0, 5, 10, 15, 30, 45, 60, 75, or 90 minutes; or 2, 3, 4, 5, 6, 12, 18, or 24 hours; or 1, 2, 3, 4, 5, 6, 7, 8, 9, 10, 12, 14, 21, or 28 days, of starting the culture.

67. The method of claim 46, wherein the expression levels are measured multiple times in a time series, e.g., at least: 2 times, 3 times, 4 times, 5 times, 6 times, 7 times, 8 times, 9 times, or 10 times.

68. The method of claim 67, wherein the multiple measurements are made over a period of about: 1 day, 2 days, 3 days, 4 days, 5 days, 6 days, or 7 days, or about: 1 week, 2 weeks, 3, or 4 weeks, or more.

69. The method of claim 46, wherein the sample from the culture comprises both cells and culture medium.

70. The method of claim 69, wherein the cells in the sample are lysed before measuring the expression levels.

71. The method of claim 70, wherein the cells in the sample are lysed by mechanical/physical methods, enzymatic methods, or chemical methods.

72. The method of claim 46, wherein the MSC culture is a monolayer on a planar solid surface.

73. The method of claim 46, wherein the MSC culture is a liquid suspension culture.

74. The method of claim 73, wherein the liquid suspension culture comprises growing the cells on a microcarrier.

75. The method of claim 74, wherein the microcarrier comprises dextran, collagen, polystyrene, glass, polymers, agarose, or a combination thereof.

76. The method of claim 73, wherein the liquid suspension culture is in a bioreactor.

77. The method of claim 76, wherein the bioreactor has a capacity of about: 1, 10, 50, 100, 250, 500, 750 mL or more, e.g., 1 L, 3 L, 5 L, 15 L, 50 L, 100 L, 250 L, 500 L, 1000 L, 5000 L, 10000 L, 20000 L, 30000 L, 40000 L, or 50000 L, or more.

78. The method of claim 73, wherein the MSCs are cultured by batch, batch refeed, fed batch, partial medium exchange or perfusion.

79. The method of claim 46, wherein the MSCs are mammalian MSCs.

80. The method of claim 79, wherein the mammalian MSCs are human MSCs.

81. The method of claim 46, wherein the culture is a working cell bank or a master cell bank.

82. The method of claim 46, further comprising the step of measuring the expression level of one or more markers of any additional tissue or organ of the mesodermal or ectodermal lineage.

83. The method of claim 46, further comprising determining if the cells in a sample of the MSC culture:
i) adhere to plastic in standard culture conditions;
ii) meet the condition that more than about: 80%, 85%, 90%, 95%, or more, of the cells express one or more of CD105, CD73, or CD90, more preferably, that 95% or more of the cells express one or more of CD105, CD73, or CD90, still more preferably, that 95% or more of the cells express one or more of CD105, CD73, and CD90;
iii) meet the condition that less than about: 10%, 9%, 8%, 7%, 6%, 5%, 4%, 3%, 2%, 1%, or fewer, of the cells express one or more of CD45; CD34; CD14 or CD11b; CD79α or CD19; or HLA-DR, more preferably, that 2% or fewer of the cells express one or more of CD45; CD34; CD14 oral CD11b; CD79a or CD19; or HLA-DR, still more preferably, that 2% or fewer of the cells express CD45; CD34; CD14 or CD11b; CD79a or CD19; and HLA-DR;
iv) are capable of differentiating along adipocyte, chondroblast, or osteoblast lineages under standard in vitro differentiating conditions; or
v) are characterized by 1, 2, 3, or all 4 of i), ii), iii), and iv).

84. The method of claim 46, further comprising visualizing the morphology of cells in a sample from the culture.

85. The method of claim 84, wherein the visualization is to classify the differentiation state of the cells by their morphology.

86. The method of claim 46, further comprising the step of providing a user-readable display of the measured expression levels.

87. The method of claim 86, wherein the display is in the form of one or more graphs of the expression levels.

88. The method of claim 87 wherein the one or more graphs of the expression levels are of a time series of expression levels.

89. The method of claim 86, wherein the display provides a summary of the differentiation state of the culture.

90. A method of treating a subject in need of cells selected from MSCs, adipocytes, chondroblasts, or osteoblasts, comprising providing the subject a therapeutically effective amount of the cells from a culture tested by the method of any one of the preceding claims and determined to contain cells in the necessary differentiation state.

91. A kit suitable for performing the method of claim 46, comprising reagents for detecting the expression level of:
one or more markers of adipogenesis selected from KLF5, FABP4, Adiponectin/ADIPOQ, Chemerin/RARRES2, KLF4, and PAI1;
one or more markers of osteogenesis selected from Runx2, ALPL, Osteocalcin/BGLAP, CTNNB1, and Osteonectin/SPARC; or
one or more markers of chondrogenesis selected from Sox9, COL2A1, MIA, and COMP.
or any combination of the markers.

92. The kit of claim 91, wherein the reagents are detectably labeled and suitable for the simultaneous singleplex or multiplex detection of the one or more markers.

93. The kit of claim 90, wherein the reagents are, for each marker, a pair of detectably labeled antibodies that specifically bind the marker.

94. The kit of claim 93, wherein the antibodies are fluorescently labeled and suitable for simultaneous multiplex detection.

95. A non-transient, computer-readable medium comprising instructions that, if executed by a processor, would cause the processor to perform steps comprising:
accepting data representing the expression levels of:
one or more markers of adipogenesis selected from KLF5, FABP4, Adiponectin/ADIPOQ, Chemerin/RARRES2, KLF4, and PAI1;
one or more markers of osteogenesis selected from Runx2, ALPL, Osteocalcin/BGLAP, CTNNB1, and Osteonectin/SPARC;
one or more markers of chondrogenesis selected from Sox9, COL2A1, MIA, and COMP; or
any combination of the markers; and
evaluating the expression level of one or more of the markers, wherein a change in the expression levels, relative to a suitable control, indicates a change in the differentiation state of the MSC culture.

96. The computer-readable medium of claim 95 suitable for performing a method of monitoring the differentiation state of a mesenchymal stem cell (MSC) culture, comprising measuring the expression level of: one or more markers of adipogenesis, one or more markers of osteogenesis, or one or more markers of chondrogenesis, in an isolated sample from the culture, wherein a change in the expression level of one or more of the markers, relative to a suitable control, indicates a change in the differentiation state of the MSC culture.

97. A system comprising the computer-readable medium of 95 and a processor for executing the instructions.

98. The system of claim 95, further comprising a user-readable display for displaying the measured gene expression levels.
